(19) United States
(12) Patent Application Publication
(10) Pub. No.: US 2014/0147846 A1
(43) Pub. Date: May 29, 2014

(54) DETECTION OF SEQUENCE VARIANTS IN THE HUMAN EPIDERMAL GROWTH FACTOR RECEPTOR (EGFR) GENE

(71) Applicants: Lawrence J. Wangh, Auburndale, MA (US); Shana Marie Tetrault, Woburn, MA (US); J. Aquiles Sanchez, Framingham, MA (US)

(72) Inventors: Lawrence J. Wangh, Auburndale, MA (US); Shana Marie Tetrault, Woburn, MA (US); J. Aquiles Sanchez, Framingham, MA (US)

(73) Assignee: BRANDEIS UNIVERSITY, Waltham, MA (US)

(21) Appl. No.: 14/119,397

(22) PCT Filed: May 25, 2012
(86) PCT No.: PCT/US2012/039594

§ 371 Date: Feb. 6, 2014

Related U.S. Application Data
(60) Provisional application No. 61/490,347, filed on May 26, 2011.

(51) Int. Cl. C12Q 1/68

(52) U.S. Cl. CPC C12Q 1/6827
USPC 435/6.11

(57) ABSTRACT

Provided herein are methods for detecting and identifying sequence variants in the human epidermal growth factor receptor (EGFR) gene, and compositions and kits for performing such methods. In particular, nucleic acid amplification and fluorescence detection methods are provided for the detection and identification of EGFR sequence variants.

### FIELD

Provided herein are methods for detecting and identifying sequence variants in the human epidermal growth factor receptor (EGFR) gene, and compositions and kits for performing such methods. In particular, nucleic acid amplification and fluorescence detection methods are provided for the detection and identification of EGFR sequence variants.

### BACKGROUND

The epidermal growth factor receptor (EGFR) is a tyrosine kinase proto-oncoprotein that phosphorylates tyrosine residues of downstream effector proteins which, in turn, trigger a cascade of events resulting in cell proliferation, motility, and survival (Wang et al. 2004; herein incorporated by reference in its entirety) (See FIG. 1 of Wang et al.). EGFR is encoded by a proto-oncogene consisting of 28 exons, located on the short arm of chromosome 7, and is a member of the c-erb B family, a group of receptor tyrosine kinases (Kondo and Shimizu 1983, Herbst 2004; herein incorporated by reference in their entireties). The protein product of the EGFR gene is 170 kd and made up of 3 domains; an extracellular ligand binding domain, a transmembrane region, and an intracellular domain that has kinase activity (Kondo and Shimizu 1983; herein incorporated by reference in its entirety). EGFR is auto regulated and is activated when its extracellular domain binds a growth ligand. Upon ligand binding the single chain EGFR can either homodimerize or heterodimerize with members of the c-erb B family. Dimerization allows for the auto-phosphorylation of EGFR on key tyrosine residues located within its tyrosine kinase domain (TKD) at the intracellular end of the protein (Zhang et al. 2006, FIG. 2; herein incorporated by reference in its entirety). Phosphorylation of EGFR's TKD allows the receptor to phosphorylate downstream proteins that lead to signal transduction events.

The ability of EGFR to induce proliferation is critical to its role as a proto-oncoprotein. A mutation within the EGFR gene sequence results in a constitutively activate onco-protein. EGFR genetic alterations are largely responsible for the development and progression of non-small cell lung carcinomas (NSCLC), the most common cause of cancer death worldwide. The mutations which constitutively activate EGFR initiate tumorigenesis, progression of lung cancer, increased tumor aggressiveness and affect treatment outcome. EGFR genetic abnormalities are strongly associated with NSCLC. Mechanisms of EGFR constitutive activation arise by receptor amplification, in-frame insertions or deletions, single nucleotide substitutions, or duplications. EGFR protein is typically found at 40,000-100,000 copies per cell, however in NSCLC, receptor amplification can result in 2×10⁶ receptors per cell (Herbst 2004; herein incorporated by reference in its entirety). The increase in protein copy number is a driving force towards metastatic progression. Eighty-four percent of squamous cell carcinomas, a subset of NSCLC, have receptor amplification leading to an increase in the growth rate of tumors. The survival rate decreases as the number of receptors increase. Therefore receptor over-expression has a very poor prognosis in NSCLC.

EGFR mutations are common in lung and anal cancer, and glioblastoma multiformes. Mutations arise in the tyrosine kinase domain (TKD) encoded by EGFR exons 18-21. Studies have shown that there are 4 sites within exons 18-21, deemed hotspots that are most likely to lock the protein into an active configuration. In addition to driving metastatic progression these activating mutations dictate responsiveness to treatment (Paez 2004; herein incorporated by reference in its entirety). Understanding the role that mutations play in treatment success has driven research for the identification of mutation that lead to drug sensitivity and those that lead to resistance. Identifying these mutations allows the development of drugs which are better suited to the abnormal EGFR that drive tumor progression. For patients who harbor mutations within the TKD the response rate is approximately 75%. There are high frequency ‘classical’ mutations in EGFR TKD that account for 90% of the mutations observed that affect treatment outcome.

Structural analysis has shown that the majority of mutations which arise in EGFR are deletion mutations located in the TKD of the protein. The important amino acid residues in this region phosphorylate downstream affecters of EGFR thereby facilitating signal transduction. Mutations that arise in this region abolish the proteins ability to inactivate itself. Although hotspots exist, mutations along the entire length of the TKD have been observed and their frequency documented.

Since EGFR over-expression and mutation allow for the establishment and progression of NSCLC they have become a specialized target for treating NSCLC. Drug development has focused on the attenuation of the receptor and its tyrosine kinase domain as a means of stopping disease progression and placing the cancer into remission. Monoclonal antibodies (MA) are targeted against the ligand binding domain of EGFR. MA's function by binding to the ligand binding domain of EGFR and reducing the rate of cell proliferation, which leads to the down regulation of the receptor (Fan 1993; herein incorporated by reference in its entirety). The limitation of MA's is that they can elicit an immune response since the antibody used is a chimera from mice and human antibodies. In the event of an immune response the MA's are destroyed and disease progression continues.

Tyrosine kinase inhibitors (TKI's), on the other hand, are small molecules that interact with the cytoplasmic domain of EGFR. They function to prevent auto-phosphorylation of EGFR by competing for the ATP cleft in the TKD (Mok et al. 2009; herein incorporated by reference in its entirety). TKI's, such as Gefitinib and Erlotinib have shown dramatic anti-tumor affects in patients with NSCLC. Further investigation into responders to TKI treatment showed that the tumors which shrank in the presence of the TKI's all harbored mutations within the TKD of EGFR, most specifically in exons 18-21. The clinical responses to these TKIs, although dramatic, have only worked on a fraction of the population. Response to TKI's is 10% in the European population and 30% in the Asian population (Gazdar 2009; herein incorporated by reference in its entirety). Sequencing analysis revealed that individuals with a significant and dramatic response to these drugs have mutations in the tyrosine kinase domain of EGFR, most specifically in exons 18-21 of the gene. Indeed, the response rate to TKI's was 75% for individuals harboring EGFR exon 18-21 mutations. This discovery suggested that mutations within this region are responsible for initiating malignant transformation. This suggested that these mutations promote malignant transformation, (Sorscher 2004, Lynch 2004; herein incorporated by reference in their entireties). Tumors harboring mutations were mostly adenocarcinomas of NSCLC, more often in women than in men, and more than likely to be in individuals of Asian origin rather than Caucasian (Paez 2004; herein incorporated by reference in its entirety). Studies were undertaken to specifically identify the mutations which determined sensitivity to TKI's. These studies showed that 45% of EGFR sensitizing mutations came from in-frame deletions in exon 19, while 40-45% of sensitization mutations came from point mutations in exon 21, specifically L858R.

The discovery of activating mutations also led to the discovery of EGFR resistance mutations to TKIs. Initially, individuals with EGFR mutations which rendered them sensitive to TKIs responded very well to the drugs; however, within 2 years all those who responded to TKIs became resistant to the drug. Upon further investigation, a secondary mutation was identified within the resistant tumors, this mutation was also found within the TKD. This mutation increases the efficiency of the TKD, and decreases progression-free survival (Maheswaran et al. 2008; herein incorporated by reference in its entirety). Furthermore, the resistance mutations were identified in samples from individuals that have been treated with TKIs, thereby elucidating the fact that T790M is a secondary mutation that arises post treatment with TKIs, (Kobayashi 2005; herein incorporated by reference in its entirety). Crystallographic studies demonstrated that the T790M mutation interferes with TKI activity through steric hindrance (Kobayashi 2005; herein incorporated by reference in its entirety). Nevertheless, TKIs worked dramatically on a subset of individuals with NSCLC and have become a very important tool in the treatment of NSCLC. These results suggest that EGFR-mutant lung cancer is a distinct class of non-small cell lung cancer.

Paez et al. has proposed that the identification of EGFR mutations in other malignancies, like glioblastomas in which EGFR alterations had previously been identified (Yamazaki et al., 1988; herein incorporated by reference in its entirety), may identify other patients who would benefit from treatment with EGFR inhibitors, rather than treating them with the unselective traditional cytotoxic therapies. Large-scale screening of patients with lung cancer for EGFR mutations is feasible and can have a role in treatment decisions (Rosell et al. 2009; herein incorporated by reference in its entirety).

Mutation identification is crucial in determining the outcome of disease and the effectiveness of a given treatment (Paez 2004; herein incorporated by reference in its entirety). A molecular marker such as EGFR mutation status can identify risk, prognosis, and treatment sensitivity (Herbst 2008; herein incorporated by reference in its entirety). Identification of mutations within EGFR prior to treatment selection would decrease both morbidity and mortality in cancer patients.

An understanding of a tumors sensitivity and resistance to conventional therapy and second line TKI therapies is necessary prior to treatment. The failure to establish EGFR status can results in poor response to treatment, increased morbidity, and death due to toxicity. Cancer treatment selection would benefit significantly from a companion diagnostic assay that could identify mutations in EGFR that would render a particular drug suitable or ineffective for a given individual.

Thus a means to identify EGFR mutations is vital for treatment selection and outcome. Knowing the molecular signature of NSCLC would prevent the needless administration of chemotherapeutics that are not likely to work and would aid in a more efficient means of treatment selection. EGFR mutation identification for treatment selection would ultimately reduce the time to remission for a patient and decrease morbidity and mortality associated with chemotherapy.

Identification of EGFR mutations would not only aid in the selection of best fit treatments for the affected individuals (personalized medicine), but would also aid in follow up care of patients. Measuring disease recurrence is often very difficult. Identification of aberrant EGFR sequences after disease remission would be indicative of disease resurgence, especially the emergence of the T790M mutation that is not currently responsive to treatment. For this reason the identification of EGFR mutations at the primary tumor site or in other organs would be an effective way of measuring and following metastases and disease recurrence.

There are currently two prominent methods used to identify EGFR mutations, DNA sequencing and immunohistchemistry (IHC). Unfortunately, current technologies for detection and characterization of EGFR mutations are expensive, laborious, and not always accurate. Although other methods for mutation detection exist for EGFR, the other techniques require prior knowledge of a mutations existence in a given sample, or they are too time intensive and are therefore not useful adjuncts to drug development or clinical practice.

Sequencing is currently the method of choice for the assessment of genetic variability. It has been used to identify insertions, deletions, and mutation in EGFR. Sequencing of the EGFR target is either carried out directly from the amplified product, or is carried out after the product is digested with a restriction enzyme and run out on a gel for the selection of aberrant bands (Robles 2010; herein incorporated by reference in its entirety). While sequencing is a very accurate method for the detecting the major genetic variants in a population of molecules, it has its limitations. First, although, sequencing generates a great deal of data, much of information is redundant, making the cost of information per sample high. Second, sequencing requires skilled labor for sample preparation. For these reason sequencing of EGFR samples is not currently practical for routine clinical use.

The IHC method of EGFR mutation identification relies on antibodies binding to specific mutated EGFR proteins, or the deletion mutations of exon 19 (Kitamura 2010; herein incorporated by reference in its entirety). The immunoreactivity that occurs when the antibody binds the protein generates a fluorescent signal that signifies the presence of the altered protein. The overall sensitivity of IHC is low, 47%, and the results are significant only when they are positive. False negatives are often recorded using this method because of post-translational modifications to the protein and detection thresholds (Kitamura 2010; herein incorporated by reference in its entirety). Therefore, although IHC for EGFR mutation detection is relatively specific at −50%, not all EGFR mutation positive patients can be identified using this method.

### SUMMARY

Provided herein are methods for detecting and identifying sequence variants in the human epidermal growth factor receptor (EGFR) gene, and compositions and kits for performing such methods. In particular, nucleic acid amplification and fluorescence detection methods are provided for the detection and identification of EGFR sequence variants.

Also provided herein are general methodologies and reagents for detection of mutations in desired target sequences. EGFR provides exemplary target sequences, but the methods and reagents may be used on any number of target sequences, and find particular use for the detection and analysis of target sequences that may carry one or more known and/or unknown mutations or other sequences of interest.

In some embodiments, provided herein are methods for identifying one or more sequences (e.g., mutations) in a target nucleic acid molecule (e.g., human EGFR gene), comprising (a) providing: (i) a sample suspected of comprising the target nucleic acid molecule (e.g., human EGFR gene), or a portion of the target nucleic acid molecule (e.g., human EGFR gene), and (ii) detection reagents comprising at least one pair of primers and at least one detectably distinguishable probe set of two hybridization probes that hybridize to adjacent target nucleic acid sequences in the target nucleic acid molecule (e.g., human EGFR gene), each probe set comprising: (A) a quencher probe labeled with a non-fluorescent quencher, and (B) a signaling probe labeled with a fluorescence-emitting fluorophore and a non-fluorescent quencher, wherein the signal probe does not emit fluorescence above background when not hybridized to its target sequence, but emits a fluorescence signal above background upon hybridization to its target sequence in the absence of bound quencher probe, wherein, if both signaling and quencher probes are hybridized to their target nucleic acid sequences, the non-fluorescent quencher of the quencher probe quenches the signal from the signaling probe; (b) amplifying nucleic acid from the target nucleic acid molecule (e.g., human EGFR gene) with the primers; (c) detecting the fluorescence of the fluorescence-emitting fluorophore from each detectably distinguishable probe set at a range of temperatures; (d) generating temperature-dependent fluorescence signatures for each fluorescence-emitting fluorophore; and (e) analyzing the temperature-dependent fluorescence signatures to detect the presence or absence of the one more sequences in the sample.

In some embodiments, the melting temperature of the signaling probe in a probe set is higher than the melting temperature of the associated quencher probe. In some embodiments, the quencher probe and/or signaling probe are configured to hybridize to a nucleic acid sequence within the target nucleic acid molecule (e.g., human EGFR gene). In some embodiments, the fluorescence-emitting fluorophore and the non-fluorescent quenchers of each probe set are capable of interacting by FRET or contact quenching. In some embodiments, the detection reagents comprise two or more probe sets. In some embodiments, two or more probe sets comprise different fluorescence-emitting fluorophores that emit at detectably different wavelengths. In some embodiments, two or more probe sets comprise the same fluorescence-emitting fluorophore. In some embodiments, the probes sets comprising the same fluorescence-emitting fluorophores hybridize to their target nucleic acid sequences at detectably different melting temperatures with their target nucleic acid sequences. In some embodiments, the each of the two or more probe sets are detectably distinguishable from all other probe sets in said detection reagents by (1) melting temperature, (2) emission wavelength of said fluorescence-emitting fluorophore, or (3) a combination thereof. In some embodiments, the each of the two or more probe sets are not detectably distinguishable from all other probe sets in said detection reagents; rather, two or more probe sets produce an overall fluorescence signature. In some embodiments, the detection reagents comprise 5 or more probe sets. In some embodiments, the detection reagents comprise 10 or more probe sets. In some embodiments, a probe set is used to detect mutations in EGFR that cause drug resistance, drug sensitivity, increased cancer severity, decreased cancer severity, increased likelihood of developing cancer, decreased likelihood of developing cancer, and/or have no known effect. In some embodiments, one or both probes of said probe set are designed to have different degrees of complementarity to their target sequences in the presence of various EGFR mutations. In some embodiments, the different degrees of complementarity result in different temperature-dependent fluorescent signatures generated by a probe set and its target sequences. In some embodiments, the different temperature dependent fluorescent signatures are used to differentiate, identify, and/or detect EGFR mutations in a sample. In some embodiments, the temperature-dependent fluorescence signature comprises a melt curve or an annealing curve. In some embodiments, the analyzing the temperature-dependent fluorescence signature comprises comparison to a previously established melting curve or annealing curve. In some embodiments, temperature-dependent fluorescence signatures are obtained for each strand of the EGFR gene. In some embodiments, analyzing is performed by a computer (e.g., a desktop computer, a portable computer, a handheld device, analytical equipment). In some embodiments, amplification is by a non-symmetric amplification method. In some embodiments, amplification is by LATE-PCR amplification. In some embodiments, the probes in at least one detectably distinguishable probe set have melting temperatures with their target nucleic acid sequences below the annealing temperature of at least one primer of the amplification reaction.

In some embodiments, one or more probe sets are configured to hybridize to a region of the human EGFR gene to detect, identify, and/or differentiate mutations in exon 18 or human EGFR. In some embodiments, one or more primer pairs are configured to amplify a region of the human EGFR gene comprising the complete exon 18, or a portion thereof. In some embodiments, one or more exon 18 probe sets comprise SEQ ID NO.:9, SEQ ID NO.:10, SEQ ID NO.:11, SEQ ID NO.:12, and/or SEQ ID NO.:13, or have 70% or greater identity therewith (e.g., 75%, 80%, 85%, 90%, 95%). In some embodiments, one or more exon 18 probe sets comprise SEQ ID NOS.:9-13, or have 70% or greater identity therewith (e.g., 75%, 80%, 85%, 90%, 95%). In some embodiments, the one or more primer pairs for the amplification of exon 18 comprise SEQ ID NO.:1 and SEQ ID NO.:2 or have 70% or greater identity therewith (e.g., 75%, 80%, 85%, 90%, 95%).

In some embodiments, one or more probe sets are configured to hybridize to a region of the human EGFR gene to detect, identify, and/or differentiate mutations in exon 19 or human EGFR. In some embodiments, one or more primer pairs are configured to amplify a region of the human EGFR gene comprising the complete exon 19, or a portion thereof. In some embodiments, one or more exon 19 probe sets comprise SEQ ID NO.:14, SEQ ID NO.:15, SEQ ID NO.:16, SEQ ID NO.:17, and/or SEQ ID NO.:18, or have 70% or greater identity therewith (e.g., 75%, 80%, 85%, 90%, 95%). In some embodiments, one or more exon 19 probe sets comprise SEQ ID NOS.:14-18, or have 70% or greater identity therewith (e.g., 75%, 80%, 85%, 90%, 95%). In some embodiments, the one or more primer pairs for the amplification of exon 19 comprise SEQ ID NO.:3 and SEQ ID NO.:4 or have 70% or greater identity therewith (e.g., 75%, 80%, 85%, 90%, 95%).

In some embodiments, one or more probe sets are configured to hybridize to a region of the human EGFR gene to detect, identify, and/or differentiate mutations in exon 20 or human EGFR. In some embodiments, one or more primer pairs are configured to amplify a region of the human EGFR gene comprising the complete exon 20, or a portion thereof. In some embodiments, one or more exon 20 probe sets comprise SEQ ID NO.:19, SEQ ID NO.:20, SEQ ID NO.:21, and/or SEQ ID NO.:22, or have 70% or greater identity therewith (e.g., 75%, 80%, 85%, 90%, 95%). In some embodiments, one or more exon 20 probe sets comprise SEQ ID NOS.: 19-22, or have 70% or greater identity therewith (e.g., 75%, 80%, 85%, 90%, 95%). In some embodiments, the one or more primer pairs for the amplification of exon 20 comprise SEQ ID NO.:5 and SEQ ID NO.:6 or have 70% or greater identity therewith (e.g., 75%, 80%, 85%, 90%, 95%).

In some embodiments, one or more probe sets are configured to hybridize to a region of the human EGFR gene to detect, identify, and/or differentiate mutations in exon 21 or human EGFR. In some embodiments, one or more primer pairs are configured to amplify a region of the human EGFR gene comprising the complete exon 18, or a portion thereof. In some embodiments, one or more exon 21 probe sets comprise SEQ ID NO.:23, SEQ ID NO.:24, SEQ ID NO.:25, SEQ ID NO.:26, SEQ ID NO.:27, SEQ ID NO.:28, and/or SEQ ID NO.:29, or have 70% or greater identity therewith (e.g., 75%, 80%, 85%, 90%, 95%). In some embodiments, one or more exon 21 probe sets comprise SEQ ID NOS.:23-29, or have 70% or greater identity therewith (e.g., 75%, 80%, 85%, 90%, 95%). In some embodiments, the one or more primer pairs for the amplification of exon 21 comprise SEQ ID NO.:7 and SEQ ID NO.:8 or have 70% or greater identity therewith (e.g., 75%, 80%, 85%, 90%, 95%).

In some embodiments, one or more probe sets comprise one or more detectably distinguishable probes. In some embodiments, one or more probe sets comprise one or more probe pairs (e.g. signal and quencher) that are detectably distinguishable from other probe sets. In some embodiments, one or more probe sets are not detectably distinguishable from other probe sets used in an assay, rather, the overall fluorescence signature is analyzed to identify mutations. In some embodiments, when detectably distinguishable probe sets are used, the overall fluorescence signature is analyzed to identify mutations.

In some embodiments, the desired target to be detected (e.g., a EGFR gene harboring a mutation of significance (e.g., a drug resistance mutation)) is present in a sample comprising a substantial amount of nucleic acid from non-target sources (e.g., non-cancer cells, non-drug-resistant cells, etc.). In some embodiments the target is present at less than 20% of the total nucleic acid in the sample (by copy number) (e.g., less than 10%, less than 5%, less than 1%, less than 0.5%, or less than 0.1%). In some embodiments, EGFR mutations are detectable in sample comprising less than 10%, less than 5%, less than 1%, less than 0.5%, or less than 0.1% target nucleic acid.

In some embodiments, the percentage of the desired target to be detected (e.g., a EGFR gene harboring a mutation of significance (e.g., a drug resistance mutation)) that is present in a sample comprising a substantial amount of nucleic acid from non-target sources (e.g., non-cancer cells, non-drug-resistant cells, etc.) is increased prior to target detection either by a selective isolation prior to amplification, or by a selective amplification process, or both. Selective isolation and/or amplification can increase the proportion of the desired target more than 2 fold, 5 fold, 10 fold, 20 fold, 100 fold, 1000 fold, 10,000 fold.

In some embodiments, provided herein are reagent kits for detecting, identifying, and/or differentiating one or more sequences in a sample comprising: (a) at least one pair of primers, wherein said primers are configured bind to regions of the target nucleic acid molecule (e.g., EGFR gene) (e.g., flanking one or more of exons 18-21), and wherein primers are configured to amplify a region of the target nucleic acid molecule (e.g., EGFR gene) (e.g. one or more of exons 18-21); and (b) at least one detectably distinguishable probe set of two hybridization probes which hybridize to adjacent target nucleic acid sequences within the target nucleic acid molecule (e.g., EGFR gene) (e.g. one or more of exons 18-21), comprising: (i) a quencher probe labeled with a non-fluorescent quencher, and (ii) a signaling probe labeled with a fluorescence-emitting fluorophore and a non-fluorescent quencher, wherein the signal probe does not emit fluorescence above background when not hybridized to its target sequence, but emits a fluorescence signal above background upon hybridization to its target sequence in the absence of bound quencher probe, wherein, if both signaling and quencher probes are hybridized to their adjacent target nucleic acid sequences, the non-fluorescent quencher of the quencher probe quenches the signal from the signaling probe. In some embodiments, the melting temperature of the signaling probe in a probe set is higher than the melting temperature of the associated quencher probe. In some embodiments, the fluorescence-emitting fluorophore and said non-fluorescent quenchers of each probe set are capable of interacting by FRET or contact quenching. In some embodiments, each probe set is detectably distinguishable from all other probe sets in said detection reagent kit by (1) melting temperature, (2) emission wavelength of said fluorescence-emitting fluorophore, or (3) a combination thereof. In some embodiments, each probe set is not detectably distinguishable from each other probe set. In some embodiments, multiple probe sets produce an overall fluorescence signature. In some embodiments, a probe set comprises a signal probe and a quencher probe. In some embodiments, a probe set comprises one or more signal probes and one or more quencher probes. In some embodiments, a probe set comprises 2, 3, 4, 5, 6, 7, 8, 9, 10, 11, 12, 13, 14, 15, 16, 17, 18, 19, 20, etc. quencher and signal probes. In some embodiments, the detection reagents comprise 5 or more probe sets. In some embodiments, the detection reagents comprise 10 or more probe sets. In some embodiments, a probe set is used to differentiate EGFR genes which confer of different drug resistances or cancer severities. In some embodiments, the primers are provided in the proper ratio for amplification by LATE-PCR. In some embodiments, probes in at least one probe set have melting temperatures with their target nucleic acid sequences below the annealing temperature of at least one primer of the amplification reaction. In some embodiments, reagent kits comprise one or more detectably distinguishable probe sets are configured to hybridize to exon 18 of the human EGFR gene. In some embodiments, reagent kits comprise one or more detectably distinguishable probe sets are configured to hybridize to exon 19 of the human EGFR gene. In some embodiments, reagent kits comprise one or more detectably distinguishable probe sets are configured to hybridize to exon 20 of the human EGFR gene. In some embodiments, reagent kits comprise one or more detectably distinguishable probe sets are configured to hybridize to exon 21 of the human EGFR gene. In some embodiments, reagent kits comprise primers and probes configured for: differentiating between wild-type EGFR and EGFR with an exon 18 mutation; (b) differentiating between wild-type EGFR and EGFR with an exon 19 mutation; (c) differentiating between wild-type EGFR and EGFR with an exon 20 mutation; (d) differentiating between wild-type EGFR and EGFR with an exon 21 mutation. In some embodiments, reagent kits comprise one or more additional oligonucleotides. In some embodiments, additional oligonucleotides are configured to suppress mis-priming during amplification reactions. In some embodiments, additional oligonucleotides are configured to disrupt structural elements within target nucleic acid sequences during amplification reactions or during probing of amplified sequences.

In some embodiments, reagent kits may comprise probe sets, primers, amplification reagents (e.g. amplification buffer, DNA polymerase, control reagents (e.g., positive and negative controls)) or any other components that are useful, necessary, or sufficient for practicing any of the methods described herein, as well as instructions, analysis software (e.g., that facilitates data collection, analysis, display, and reporting), computing devices, instruments, or other systems or components.

In some embodiments, provided herein is a homogeneous assay method for analyzing at least one single-stranded nucleic acid target sequence in a sample, comprising: (a) providing a sample comprising at least one nucleic acid target sequence in single-stranded form and for each nucleic acid target sequence a set of at least two interacting hybridization probes, each of which hybridizes to the at least one target, comprising: (i) at least one quencher probe labeled with a non-fluorescent quencher, and (ii) at least one signaling probe that upon hybridization to the at least one target sequence in the sample in the absence of the quencher probe emits a signal above background, wherein, if both a quencher and signaling probe are hybridized to the at least one target sequence, the non-fluorescent quencher of the quencher probe quenches the signal from the signaling probe; and (b) analyzing hybridization of the signaling and quenching probes to the at least one target sequence as a function of temperature, the analysis including an effect on each signaling probe due to its associated quencher probe, including but not limited to analyzing signal increase, signal decrease, or both, from each signaling probe.

In some embodiments, provided herein are methods for detecting mutations in the human EGFR gene, comprising: (a) providing: (i) a sample suspected of comprising the human EGFR gene, or a portion of the human EGFR gene, and (ii) detection reagents comprising primer pairs for amplification of one or more of exon 18, exon 19, exon 20, and exon 21 of the human EGFR gene and one detectably distinguishable probe set corresponding to each primer pair, wherein each probe set comprises two or more hybridization probes which hybridize to adjacent target nucleic acid sequences in the human EGFR gene, each probe set comprising: (A) one or more quencher probes labeled with a non-fluorescent quencher, and (B) one or more signaling probes labeled with a fluorescence-emitting fluorophore and a non-fluorescent quencher, wherein said signaling probes do not emit fluorescence above background when not hybridized to the target sequence, but emit a fluorescence signal above background upon hybridization to the target sequence in the absence of an adjacently bound quencher probe, wherein, if both a signaling probe and an adjacently-bound quencher probe are hybridized to their adjacent target nucleic acid sequences, the non-fluorescent quencher of the quencher probe quenches the signal from the signaling probe; (b) amplifying one or more of exon 18, exon 19, exon 20, and exon 21 of the human EGFR gene with said primers; (c) detecting the fluorescence of said fluorescence-emitting fluorophore from each detectably distinguishable probe set at a range of temperatures; (d) generating temperature-dependent fluorescence signatures for each detectably distinguishable probe set; and (e) analyzing said temperature-dependent fluorescence signatures to detect the presence or absence of one or more mutations in one or more of exon 18, exon 19, exon 20, and exon 21 of the human EGFR gene. In some embodiments, primer pairs comprise primer pairs for amplification of EGFR exon 18, exon 19, exon 20, and exon 21. In some embodiments, primer pairs comprise SEQ ID NO.:1 and SEQ ID NO.:2, SEQ ID NO.:3 and SEQ ID NO.:4, SEQ ID NO.:5 and SEQ ID NO.:6, and SEQ ID NO.:7 and SEQ ID NO.:8. In some embodiments, probe sets are configured to hybridize to each of exon 18, exon 19, exon 20, and exon 21. In some embodiments, probe sets comprise SEQ ID NOS.:9-13, SEQ ID NOS.:14-18, SEQ ID NOS.: 19-22, and SEQ ID NOS.:23-29. In some embodiments, probes hybridize to the entire sequence of the amplicons of exon 18, exon 19, exon 20, and exon 21 of the human EGFR gene.

In some embodiments, provided herein are reagent kits comprising: (a) primer pairs for the amplification of exon 18, exon 19, exon 20, and exon 21 of the human EGFR gene, and (b) probe sets configured to hybridize to amplicons produced from exon 18, exon 19, exon 20, and exon 21 of the human EGFR gene. In some embodiments, reagent kits comprise primers of SEQ ID NOS: 1-8. In some embodiments, reagent kits comprise probes of SEQ ID NOS:9-29.

In some embodiments, signaling probes comprise quenched fluorophores. In some embodiments, the melting temperature of the signaling probe in a probe set is higher than the melting temperature of an associated quenching probe.

In some embodiments, methods provided herein are performed in a single reaction vessel. In some embodiments, methods provided herein are performed in single-vessel (e.g., tube, well, etc.) screening assays to identify, detect, and/or differentiate mutations in a target nucleic acid present in a sample comprising multiple different target (e.g., different EGFR sequences) and/or non-target nucleic acids. In some embodiments, a sample of target sequence in single-stranded form is generated by an amplification method that generates single-stranded amplicons, for example, a non-symmetric polymerase chain reaction (PCR) method, most preferably LATE-PCR. In some embodiments, the primers (e.g., a pair of primers for each of exon 18, 19, 20, and 21) and at least one set of signaling and quencher probes (e.g., two sets, three sets, etc.) are included in the amplification reaction mixture.

In some embodiments, probe sets (e.g. one or more signaling and one or more quencher probes) are configured to hybridize to the EGFR gene sequence and to differentiate between EGFR sequences comprising different mutations (e.g. in a single sample or mixture). In some embodiments, probes hybridize with different Tₘ to different mutant sequences. In some embodiment, one or both probes of a probe set (e.g. signaling and/or quencher probes) are designed to have different degrees of complementarity to the target sequences. In some embodiments, a signaling probe and/or quencher probe is configured to hybridize to a target sequence with different degree of complementarity to different mutant sequences (e.g. with different Tₘ to the different target sequences).

In some embodiments, primers and probes are provided for use in the methods provided herein. In some embodiments, primers provided herein include: SEQ ID NOs: 1, 2, 3, 4, 5, 6, 7, and/or 8, portions thereof, and sequences complementary thereto. In some embodiments, primers provided herein include oligoncleotides with 70% or greater sequence identity with SEQ ID NOs: 1, 2, 3, 4, 5, 6, 7, and/or 8, (e.g. an oligonucleotide with 70% . . . 75% . . . 80% . . . 90% . . . 95% . . . 98% . . . 99% sequence identity), portions thereof, and sequences complementary thereto. In some embodiments, primers are provided that function substantially similarly to primers provided herein. In some embodiments, probes provided herein include: SEQ ID NOs: 9, 10, 11, 12, 13, 14, 15, 1, 6, 17, 18, 19, 20, 21, 22, 23, 24 25, 26, 27, 28, 29, portions thereof, and sequences complementary thereto. In some embodiments, probes provided herein include oligoncleotides with 70% or greater sequence identity with SEQ ID NOs: 9, 10, 11, 12, 13, 14, 15, 1, 6, 17, 18, 19, 20, 21, 22, 23, 24 25, 26, 27, 28, 29, portions thereof, and sequences complementary thereto. In some embodiments, probes are provided that function substantially similarly to probes provided herein. In some embodiments, target sequences for primers and probes provided herein comprise: the human EGFR gene, exon 18 of the human EGFR gene, exon 19 of the human EGFR gene exon 20 of the human EGFR gene exon 21 of the human EGFR gene, Genbank Accession number NG_{—}007726.2 (GI:383387805), portions thereof, and sequences complementary thereto.

In some embodiments, probing and analysis methods provided herein apply to samples containing single-stranded EGFR gene sequences, or portions thereof. Methods include analysis of a single sequence, analysis of two or more sequences in the same strand, analysis of sequences in different strands, and to combinations of the foregoing. A single-stranded nucleic acid target sequence may be a control sequence added to a sample. A nucleic acid target sequence may be DNA, RNA or a mixture of DNA and RNA. It may come from any source. For example, it may occur naturally, or the target sequence may occur in double-stranded form, in which case the single-stranded target sequence is obtained by strand separation and purification. If the single-stranded nucleic acid target sequence is a cDNA sequence, it is obtained from an RNA source by reverse transcription. In some embodiments, both strands of an EGFR gene are analyzed.

In many instances a natural source will not contain a target sequence in sufficient copy number for probing and analysis. In such instances the single-stranded target sequence is obtained by amplification, generally an amplification method that includes exponential amplification. In some embodiments an amplification reaction generates the single-stranded nucleic acid target sequence directly. In some embodiments an amplification reaction generates the target sequence in double-stranded form, in which event the single-stranded target sequence is obtained by strand separation and purification. Useful amplification methods that may be employed include, the polymerase chain reaction (PCR), including symmetric PCR, asymmetric PCR and non-symmetric LATE-PCR, any of which can be combined with reverse transcription for amplifying RNA sequences, NASBA, SDA, TMA, and rolling circle amplification. If the single-stranded nucleic acid target sequence is a cDNA sequence, the amplification method will include reverse transcription, for example, RT-PCR. In some embodiments, when non-symmetric amplification is utilized (e.g. LATE-PCR), probe sets are included in the amplification reaction mixture prior to amplification to avoid contamination.

In some embodiments, probe sets useful in methods provided herein include a signaling probe and an associated quencher probe. In some embodiments, probe sets useful in methods provided herein include one or more signaling probes and one or more associated quencher probes. The signaling probe is a hybridization probe that emits a detectable signal, preferably a fluorescent signal, when it hybridizes to a single-stranded nucleic acid target sequence in a sample, wherein the signal is quenchable by the associated quencher probe. The quencher probe does not emit visible light energy. Generally, a signaling probe has a covalently bound fluorescent moiety. Signaling probes include probes labeled with fluorophores or other fluorescent moieties, for example, quantum dots. In some embodiments, fluorophore-labeled probes are preferred. One type of signaling probe is a ResonSense® probe. A ResonSense® probe is a single-stranded oligonucleotide labeled with a fluorophore that accepts fluorescence from a DNA dye and for detection and reemits energy at a longer wavelength. Use of a ResonSense® probe involves use of a double-stranded DNA dye, a molecule that becomes fluorescent when it associates with double-stranded DNA, which in this case is the hybrid formed when the probe hybridizes to the single-stranded nucleic acid target sequence. For use of a ResonSense® probe, a DNA dye, for example, SYBR Green or SYBR Gold, is included in the sample containing the single-stranded nucleic acid target sequence along with the probe set or sets. In some embodiments, the reaction system comprises one or more detectable fluorophores. In some embodiments, one or more detectable fluorophores are attached to oligonucleotide probes. In some embodiments, analysis includes exciting the dye and detection emission from the ResonSense® probe or probes. Unbound signaling probes need not be removed, because they are not directly excited and remain single-stranded. In some embodiments, preferred signaling probes are dual-labeled probes comprised of a fluorophore and a quencher. However, it is also possible to utilize single-labelled fluorescent probes that emit little or no signal when in solution, even if stimulated, but that do fluoresce when they hybridize to a single-stranded nucleic acid sequence in a sample being analyzed. Yin-Yang probes are signaling probes of this design. In solution, a Yin-Yang probe is a double-stranded probe containing a fluorophore on one strand and an interacting non-fluorescent quencher on the other strand whose Tm is lower than the fluorescently labeled strand. When a Yin-Yang probe is in solution at the detection temperature, the fluorophore is quenched. Because the Tm of the complementary stand with the quencher is lower than the Tm of the fully complementary target strand, the fluorophore-labeled strand preferentially hybridizes to the single-stranded nucleic acid target when the detection temperature is lowered. Consequently, probe/target hybrids emit a detectable signal. Signaling probes for some embodiments provided herein are molecular beacon probes, single-stranded hairpin-forming oligonucleotides bearing a fluorescer, typically a fluorophore, on one end, and a quencher, typically a non-fluorescent chromophore, on the other end. In some embodiments, provided herein are single stranded oligonucleotides with any suitable type of secondary structure, bearing a fluorescence-emitting fluorophore on one end and a quencher on the other end (molecular-beacon-type probes). Various signaling probes for use in embodiments herein comprise varying degrees of secondary structure (e.g. different lengths of hairpin (e.g. 2 base pairs, 3, base pairs, 4 base pairs, 5 base pairs, etc.). When molecular beacon probes, and other similar types of probes, are in solution, they assume a conformation wherein the quencher interacts with the fluorescent moiety, and the probe is dark (e.g. hairpin conformation, closed conformation). When the probe hybridizes to its target, however, it is forced into an open conformation in which the fluorescent moiety is separated from the quencher, and the probe signals.

In some embodiments, one quencher probe is a quencher for one signaling probe that hybridizes adjacent to the quencher on a target. In some embodiments, one quencher probe is a quencher for two signaling probes that hybridize adjacent to the quencher (i.e. on opposite ends) on a target. In some embodiments, a signaling probe has a quencher on one end and is a quencher for another signaling probe that hybridizes adjacent to the first signaling probe on a target. In some embodiments, multiple quencher and signaling probes hybridize at adjacent positions along a target sequence, thereby providing quenchers (e.g., from a quencher probe or a signaling probe) for each signaling probe. In some embodiments, an entire target region is interrogated by probes (e.g., quencher probes and signaling probes) hybridized adjacently along the length of the target.

In quenched signaling probes, quenching may be achieved by any mechanism, typically by FRET (Fluoresence Resonance Energy Transfer) between a fluorophore and a non-fluorescent quenching moiety or by contact quenching. In some embodiments, preferred signaling probes are dark or very nearly dark in solution to minimize background fluorescence. Contact quenching more generally achieves this objective, although FRET quenching is adequate with some fluorophore-quencher combinations and probe constructions.

The quencher probe of a probe set consists of a nucleic acid strand comprising a non-fluorescent quencher. In some embodiments, the quencher is, for example, a non-fluorescent chromophore such a dabcyl or a Black Hole Quencher (Black Hole Quenchers, available from Biosearch Technologies, are a suite of quenchers, one or another of which is recommended by the manufacturer for use with a particular fluorophore). In some embodiments, preferred quenching probes include a non-fluorescent chromophore. In some embodiments, quenchers are Black Hole Quenchers. The quencher probe of a set hybridizes to the single-stranded nucleic acid target sequence adjacent to or near the signaling probe such that when both are hybridized, the quencher probe quenches, or renders dark, the signaling probe. Quenching may be by fluorescence resonance energy transfer (FRET) or by touching (“collisional quenching” or “contact quenching”).

FIG. 1 depicts an embodiment that illustrates the functioning of probe sets in analytical methods provided herein. In this embodiment there are two probe sets, probes 2, 4 and probes 6, 8. Probe 2 is a signaling probe, a molecular-beacon-type probe bearing fluorophore 3. Probe 6 is also a signaling probe, a molecular-beacon-type probe bearing fluorophore 7. Fluorophores 3, 7 are the same. Probes 4, 8 are quencher probes labeled only with Black Hole Quenchers 5 and 9, respectively. The melting temperatures (Tm's) of the probe-target hybrids (probes hybridized to single-stranded nucleic acid target sequence 1) are as follows: Tₘ probe 2>Tₘ probe 4>Tₘ probe 6>Tₘ probe 8. As the temperature of the sample is lowered from a high temperature at which no probes are bound, probes 2, 4, 6 and 8 bind to single-stranded nucleic acid target sequence 1 according to their hybridization characteristics. Probe 2, a signaling probe, binds first. 1, Panel A depicts probe 2 hybridized to sequence 1. As the temperature of the sample continues to be lowered, quencher probe 4 binds next, adjacent to probe 2 such that quencher 5 and fluorophore 3 are near to one another or touching. FIG. 1, Panel B depicts probe 4 hybridized to single-stranded nucleic acid sequence 1 adjacent to probe 2. At this point probe 2 is dark, or at least nearly dark. If, however, signaling probe 6 has begun to bind, it will emit fluorescence independently of probes 2, 4. FIG. 1, Panel C depicts probe 6 hybridized to single-stranded target sequence 1 adjacent to probe 4. Finally as the temperature continues to be lowered, probe 8 will bind, and its quencher 9 will quench fluorescence emission from fluorophore 7 of probe 6. FIG. 1, Panel D depicts probe 8 hybridized adjacent to probe 6. Analysis by hybridization is shown in FIG. 1, Panel E, which depicts the increase and decrease of fluorescence from fluorophores 3, 7 as a function of temperature. Such curves can be obtained as annealing (hybridization) curves as the temperature is lowered, or can be obtained as melting curves as the temperature is increased. As the sample temperature is lowered from 70° C., fluorescence curve 10 in Panel E first rises as probe 2 hybridizes to single-stranded nucleic acid sequence 1, then decreases as probe 4 binds, then increases again as probe 6 hybridizes, and finally decreases to a very low level as probe 8 hybridizes. One can deduce from curve 10 that each signaling probe has a higher Tₘ than its associated quencher probe.

Signaling and quenching probes useful in methods provided herein are typically mismatch tolerant (capable of hybridizing to single-stranded nucleic acid target sequences containing one or more mismatched nucleotides, or deletions or additions). In some embodiments, EGFR mutations are differentiated by the unique temperature-dependent fluorescence signatures generated by mismatches between probes and target sequences. In some embodiments, probes may be allele-specific (capable of hybridizing only to a perfectly complementary single-stranded nucleic acid target sequence in the method). In some embodiments, one probe of a set may be allele-specific; and the other probe, mismatch tolerant.

Secondary structure or sequences of a target strand outside the sequences to which probes hybridize can affect the results of annealing or melting analysis. Accordingly, in some embodiments, not every nucleotide in a nucleic acid target sequence needs to be hybridized to a probe. For example, in some embodiments, if the target sequence contains a hairpin, the corresponding probe can be designed in some cases to hybridize across the base of the hairpin, excluding the hairpin sequence. In other embodiments, every nucleotide in a target sequence, regardless of secondary structure, is hybridized to a probe. For example, in some embodiments, probes or other oligonucleotides are provided that disrupt secondary structure elements in order to allow for continuous probe-target hybridization.

In some embodiments, both the signaling and quencher probes of a probe set are mismatch tolerant. In some embodiments, a probe set may include an allele-specific signaling probe and an allele-specific quencher probe, a mismatch-tolerant signaling probe and a mismatch-tolerant quencher probe, an allele-specific signaling probe and a mismatch-tolerant quencher probe, or a mismatch-tolerant signaling probe and an allele-specific quencher probe. A mismatch-tolerant probe may be perfectly complementary to one variant of a variable target sequence, or it may be a consensus probe that is not perfectly complementary to any variant. Multiple probe sets may include combinations of sets of any of the foregoing types. A probe set with more than two probes may comprise any combination of the above-mentioned probe types. Additionally, analytical methods provided herein may utilize one or more signaling/quenching probe sets in combination with one or more conventional probes that signal upon hybridization to their target, for example, molecular beacon probes.

In some embodiments, unlabeled oligonucleotides configured to bind to regions at or near the target sequences for primers, signaling probes, or quencher probes are used. In some embodiments, these “silent probes” disrupt secondary structure within or near the target sequences and assist other probes in binding to target sequences at suitable Tₘ for subsequent analysis. In some embodiments, unlabeled oligonucleotides, which serve as “openers” of structural elements (e.g. secondary structural elements), are provided.

Probes useful in the methods provided herein may be DNA, RNA, or a combination of DNA and RNA. They may include non-natural nucleotides, for example, PNA, LNA, or 2′ o-methyl ribonucleotides. They may include non-natural internucleotide linkages, for example, phosphorothioate linkages. The length of a particular probe depends upon its desired melting temperature, whether it is to be allele-specific or mismatch tolerant, and its composition, for example, the GC content of a DNA probe.

In some embodiments, each signaling probe has a separate quenching probe associated with it. In some embodiments, one probe may be a part of two probe sets. For example, a quencher probe may be labeled with a quencher at each end, whereby the ends interact with different signaling probes, in which case three probes comprise two probe sets. Also, some embodiments may utilize both ends of a quenched signaling probe, for example, a molecular beacon signaling probe having a fluorophore on one end and a quencher on the other end. The fluorophore interacts with a quencher probe, comprising one set, and the quencher interacts with a signaling probe, comprising another set. In some embodiments, a probe set comprises more than two probes. In some embodiments, a probe set comprises a series of quencher and signaling probes which hybridize at adjacent position along a target sequence. In some embodiments, probes within a probe set may function as: quenchers for two signaling probes, a signaling probe and a quencher, a signaling probe only, a single quencher, etc. In some embodiments, a single probe set (e.g., of two or more probes) hybridizes to the entire length of a target sequence. In some embodiments, multiple probe sets, each comprising two or more probes, hybridize along the length of a target sequence (e.g., exon 18, 19, 20, and/or 21 of EGFR). In doing so, the probes canvass the entire target sequence and permit the identification and analysis of any one or more known or unknown sequences (e.g., mutations) anywhere in the target sequence. Such techniques find particular use for the analysis of target sequences that contain many mutations and/or that have high variability in the population and/or that have high rates of mutation generation.

In some embodiments, for analysis of a sample that may contain one or more sequences within the target region (e.g., wild-type and a mutant, two or more different mutants, etc.), the probe sets that are used are detectably distinguishable, for example by emission wavelength (color) or melting temperature (Tₘ). Making a probe set distinguishable by Tₘ from other probe sets is accomplished in any suitable way. For example, in some embodiments, all signaling probes in an assay have different Tₘ's. Alternatively, in some embodiments, all signaling probes have the same Tₘ, but the quencher probes have different Tₘ's. In some embodiments, probe sets are distinguishable by a combination of the signaling probe Tₘ and quenching probe Tₘ. Fluorescence detectors can commonly resolve 1-10 differently colored fluorophores. Therefore assays utilizing method provided herein can make use of up to 10 fluorophores (e.g. 1, 2, 3, 4, 5, 6, 7, 8, 9, 10, or more if fluorescence detectors allow). The same fluorescence emitter, for example, the same fluorophore, can be used on more than one signaling probe for a sample, if the signaling probe's can be differentiated for detection by their melting temperatures. In assays provided herein, Tₘ's are preferentially separated by at least 2° C., preferably by at least 5° C. and, in certain embodiments by at least 10° C. However as one skilled in the art will appreciate, Tₘ describes the equilibrium temperature of probe/target hybridization, not the rate of probe/target binding or melting. Thus, it is possible to distinguish the signaling of probes with Tₘ's of less than 2° C. because probes with the same Tm can still differ in terms of the rates at which they bind to/melt off of their respective targets. Available temperature space constrains the use of multiple signaling probes having the same fluorophore. If an assay is designed for annealing and/or melt analysis over a range of 80° C. to 20° C., for example, one can utilize more probe sets sharing a color than one can use in an assay designed for such analysis over a range of 70° C. to 40° C., for which one may be able to use only 3-5 probe sets sharing a color. Using four colors and only two probe sets sharing each color, a four-color detector becomes equivalent to an eight-color detector used with eight probes distinguishable by color only. Use of three probe sets sharing each of four colors, twelve different probes sets become distinguishable.

In some embodiments, quencher probes have lower Tₘ's than their associated signaling probes. With that relationship, the signaling probe emits a temperature-dependent signal through the annealing temperature range of both probes of the set as the temperature of the solution is lowered for an annealing curve analysis, and through the melting temperature range of both probes of the set as the temperature of the solution is raised for a melting curve analysis. If, on the other hand, the quencher probe of a probe set has a higher Tₘ than its associated signaling probe, the signaling probe's emission is quenched through the annealing temperature range and melting temperature range of both probes of the set, and no fluorescent signal is emitted for detection. This can be ascertained by examination of the annealing curve or the melting curve. The lack of signal provides less information about the single-stranded nucleic acid target sequence than does a curve of the probe's fluorescence as a function of temperature. In some embodiments, when mismatch-tolerant probes are used for analysis of a variable sequence, quencher probes with lower Tₘ's than their associated signaling probes are used with respect to all or all but one of the target sequence variants. If a quencher probe has a higher Tₘ against only one variant, signal failure will reveal that variant, as long as failure of the sample to include the single-stranded nucleic acid target sequence (particularly failure of an amplification reaction) is otherwise accounted for by a control or by another probe set for the single-stranded nucleic acid target sequence. Similarly, if not all variants are known, such signal failure will reveal the presence of an unknown variant. In some embodiments, it is preferred that in an assay utilizing multiple probe sets for at least one nucleic acid target sequence, the quencher probe of at least one probe set has a lower Tₘ than its associated signaling probe.

Melting temperature, Tₘ, means the temperature at which a nucleic acid hybrid, for example, a probe-target hybrid or primer-target hybrid, is 50% double-stranded and 50% single-stranded. For a particular assay the relevant Tₘ's may be measured. Tₘ's may also be calculated utilizing known techniques. In some embodiments, preferred techniques are based on the “nearest neighbor” method (Santa Lucia, J. (1998), PNAS (USA) 95: 1460-1465; and Allawi, H. T. and Santa Lucia, J. (1997), Biochem. 36: 10581-10594). Computer programs utilizing the “nearest neighbor” formula are available for use in calculating probe and primer Tₘ's against perfectly complementary target sequences and against mismatched target sequences. In this application the Tₘ of a primer or probe is sometimes given with respect to an identified sequence to which it hybridizes. However, if such a sequence is not given, for mismatch-tolerant probes that are perfectly complementary to one variant of a single-stranded nucleic acid target sequence, the Tₘ is the Tₘ against the perfectly complementary variant. In many embodiments there will be a target sequence that is perfectly complementary to the probe. However, methods may utilize one or more mismatch-tolerant primer or probes that are “consensus primers” or “consensus probes.” A consensus primer or probe is a primer or probe that is not complementary to any variant target sequence or, if not all possible target sequences are, to any expected or known sequence. A consensus primer is useful to prime multiple variants of a target sequence at a chosen amplification annealing temperature. A consensus probe is useful to shrink the temperature space needed for analysis of multiple variants. For a consensus primer or probe, if no corresponding target sequence is given, the Tₘ refers to the highest Tₘ against known variants, which allows for the possibility that an unknown variant may be more complementary to the primer or probe and, thus, have higher primer-target Tₘ or probe-target Tₘ.

Assays provided herein may utilize probe concentrations that are greater than or less than target nucleic acid concentration. The probe concentrations are known on the basis of information provided by the probe manufacturer or determined by the user. In the case of target sequences that are not amplified, target concentrations are known on the basis of direct or indirect counting of the number of cells, nuclei, chromosomes, or molecules are known to be present in the sample, as well as by knowing the expected number of targets sequences usually present per cell, nucleus, chromosome, or molecule. In the case of target sequences that are amplified, there are a number of ways to establish how many copies of a target sequence have been generated over the course of an amplification reaction. For example, in the case of a LATE-PCR amplification reaction the number of single-stranded amplicons can be calculated as follows: using a signaling probe without a quencher (in the case of quenched signaling probe that means the probe minus the quencher) in a limiting concentration such as 50 nM and its corresponding quencher probe in excess amount such as 150 nM, the number of cycles it takes to decrease the fluorescence to zero (or, in practical terms, to its minimal background level) is proportional to the rate of amplification of single-stranded amplicons. When fluorescence reaches zero (minimal background level), all of the signaling probes have found their target, and the concentration of the amplicons exceeds that of the signaling probe. In certain embodiments an amplification reaction may be continued until the amplicon being produced reaches a “terminal concentration.” Experiments conducted during development of embodiments provided herein demonstrated that a LATE-PCR amplification begun with differing amounts of target tends to produce eventually the same maximum concentration of amplicon (the “terminal concentration”), even though amplification begun with a high starting amount of target reaches that maximum in fewer cycles than does the amplification begun with a low starting amount of target. To achieve the terminal concentration beginning with a low amount of target may require extending the amplification through 70 or even 80 cycles.

Some embodiments utilize probe sets in which the concentration of the signaling probe is lower than the concentration of its associated quencher probe. This ensures that, when both probes are hybridized to their at least one nucleic acid target sequence, the signaling probe is quenched to the greatest possible degree, thereby minimizing background fluorescence. It will be appreciated that background fluorescence in an assay is the cumulated background of each signaling probe of a given color and that probes of a different color may contribute further to background signal.

Methods provided herein include analyzing the hybridization of probe sets to single-stranded EGFR nucleic acid target sequences. In methods provided herein, hybridization of signaling probes and quencher probes as a function of temperature are analyzed for the purpose of identifying, characterizing or otherwise analyzing at least one EGFR nucleic acid target sequence in a sample. In some embodiments analysis includes obtaining a curve or, if multiple colors are used, curves of signals from signaling probes as the temperature of a sample is lowered (see FIG. 1, Panel E) or obtaining a curve or curves of signals as the sample temperature is raised, or both. It is known that the shapes of the two types of curves are not necessarily identical due to secondary structures. Either or both of those curves can be compared to a previously established curve for a known single-stranded nucleic acid target sequence as part of the analysis, for example, identifying the single-stranded nucleic acid target sequence being probed. Derivative curves can also be utilized to obtain, for example, the Tₘ of a signaling probe against a nucleic acid target sequence. It is not always necessary, and it may not be desirable, to utilize entire fluorescence curves or their derivatives. In certain embodiments analysis of the hybridization of signaling probes and quencher probes includes obtaining fluorescence readings at one or several temperatures as the sample temperature is lowered or raised, where those readings reflect an effect on each signaling probe due to its associated quencher probe. For example, if it is desired to distinguish among known variants of a target sequence, and one learns from hybridization curves of variants that fluorescence at two temperatures distinguishes the variants, one need acquire fluorescence at only those two temperatures for either direct comparison or for calculation of ratios that can be compared. In most embodiments the analysis will include signal increase, signal decrease, or both, from each signaling probe.

In some embodiments, fluorescence readings using a particular probe set over a range of temperatures generates a temperature-dependent fluorescence signature. A temperature-dependent fluorescence signature may comprise curves, data points, peaks, or other means of displaying and/or analyzing an assay or sample. In some embodiments, analysis of temperature-dependent fluorescence signatures detects, identifies, and/or differentiates different EGFR sequences (e.g. wild-type and different mutant sequences). In some embodiments, analysis is performed by a user. In some embodiments, analysis is performed by analysis software on a computer or other device.

In some embodiments, a probe set comprising two or more probes hybridizes to a target sequence such that the entire target region is interrogated by the probes. In some embodiments, probes (e.g., quencher probes and signaling probes) hybridize end-to-end along the length of a target sequence (e.g. exon 18, 19, 20, or 21 of EGFR), thereby “tiling” the target region and canvassing all sequence variations within the target region. In some embodiments, in a tiling procedure, each fluorophore is paired with a quencher as the probes hybridize to the adjacent sites along the target sequence.

In some embodiments, a probe set comprises multiple quencher and signaling probes designed to interrogate the entirety of a target region sequence (e.g. exon 18, 19, 20, or 21 of EGFR). In some embodiments, all the signaling probes of a probe set are labeled with the same fluorophore. As such, the fluorescent signature of probe set hybridizing to the target region reflect the binding/melting events of all of the probes (e.g. quencher and signaling probes) in a probe set. In some embodiments, the overall “shape” of the fluorescence signature is analyzed in order to detect, identify, and/or discriminate mutations within the target region (e.g. exon 18, 19, 20, or 21 of EGFR). In some embodiments computer data analysis methods are utilized to analyze the shape of the fluorescence signature and detect, identify, and/or discriminate mutations within the target region (e.g. exon 18, 19, 20, or 21 of EGFR). In some embodiments, multiple probe sets, each comprising a plurality of quencher and signaling probes, are labeling with detectably different signaling fluorophores in order to interrogate multiple target sequences within a single tube (e.g. each probe set interrogates a different target sequence).

Mutations may be present in one or both alleles in a cells genome. In some embodiments, the shape of a fluorescence signature is analyzed to determine whether a mutation is homozygously present, homozygously absent, or heterozygous. In some embodiments homozygous and heterozygous mutations are differentiated by the magnitude of the change in the resulting fluorescence signatures.

In some embodiments, methods provided herein include nucleic acid amplification. Some preferred methods are those which generate the target sequence or sequences in single-stranded form. LATE-PCR amplification of DNA sequences or RNA sequences (RT-LATE-PCR) is especially preferred in some embodiments. LATE-PCR amplifications and amplification assays are described in, for example, European patent EP 1,468,114 and corresponding U.S. Pat. No. 7,198,897; published European patent application EP 1805199 A2; Sanchez et al. (2004) Proc. Nat. Acad. Sci. (USA) 101: 1933-1938; and Pierce et al. (2005) Proc. Natl. Acad. Sci. (USA) 102: 8609-8614. All of these references are hereby incorporated by reference in their entireties. LATE-PCR is a non-symmetric DNA amplification method employing the polymerase chain reaction (PCR) process utilizing one oligonucleotide primer (the “Excess Primer”) in at least five-fold excess with respect to the other primer (the “Limiting Primer”), which itself is utilized at low concentration, up to 200 nM, so as to be exhausted in roughly sufficient PCR cycles to produce fluorescently detectable double-stranded amplicon. After the Limiting Primer is exhausted, amplification continues for a desired number of cycles to produce single-stranded product using only the Excess Primer, referred to herein as the Excess Primer strand. LATE-PCR takes into account the concentration-adjusted melting temperature of the Limiting Primer at the start of amplification. Tm_{[0]}^{L}, the concentration-adjusted melting temperature of the Excess Primer at the start of amplification, Tm_{[0]}^{X}, and the melting temperature of the single-stranded amplification product (“amplicon”), Tm_{A}. For LATE-PCR primers, Tm_{[0]} can be determined empirically, as is necessary when non-natural nucleotides are used, or calculated according to the “nearest neighbor” method (Santa Lucia, J. (1998), PNAS (USA) 95: 1460-1465; and Allawi, H. T. and Santa Lucia, J. (1997), Biochem. 36: 10581-10594) using a salt concentration adjustment, which in our amplifications is generally 0.07 M monovalent cation concentration. For LATE-PCR the melting temperature of the amplicon is calculated utilizing the formula: Tₘ=81.5+0.41 (% G+% C)−500/L+16.6 log [M]/(1+0.7 [M]), where L is the length in nucleotides and [M] is the molar concentration of monovalent cations. Melting temperatures of linear, or random-coil, probes can be calculated as for primers. Melting temperatures of structured probes, for example molecular beacon probes, can be determined empirically or can be approximated as the Tₘ of the portion (the loop or the loop plus a portion of the stem) that hybridizes to the amplicon. In a LATE-PCR amplification reaction Tm_{m[0]}^{L} is preferably not more than 5° C. below Tm_{[0]}^{X}, more preferably at least as high and even more preferably 3-10° C. higher, and Tm_{A} is preferably not more than 25° C. higher than Tm_{[0]}^{X}, and for some preferred embodiments preferably not more than about 18° C. higher.

LATE-PCR is a non-symmetric PCR amplification that, among other advantages, provides a large “temperature space” in which actions may be taken. See WO 03/054233 and Sanchez et al. (2004), cited above. Certain embodiments of LATE-PCR amplifications include the use of hybridization probes, in this case sets of signaling and quencher probes, whose Tm's are below, more preferably at least 5° C. below, the mean primer annealing temperature during exponential amplification after the first few cycles. Sets of signaling and quencher probes are included in LATE-PCR amplification mixtures prior to the start of amplification. A DNA fluorophore, if used, can also be incorporated into the reaction mixture prior to the start of amplification.

In some embodiments, multiple amplicons are produced from multiple primer sets in a single reaction vessel (e.g., exon 18 amplicon, exon 19 amplicon, exon 20 amplicon, exon 21 amplicon, etc.). In some embodiments, amplicons are produced from target sequences that are adjacent on a template nucleic acid. In some embodiments, oligonucleotide blockers are used to prevent extension of one amplicon into an adjacent amplicon. In some embodiments, blockers comprise one or more on-natural nucleotides, PNA, LNA, 2′ o-methyl ribonucleotides, phosphorothioates, etc. In some embodiments, blockers between amplicons (e.g. EGFR exons) inhibit extension of the upstream strand thru the intervening sequence (e.g., EGFR introns). In some embodiments, a blocker is designed to bind right at the beginning of the sequence (e.g., intron) immediately following the sequence to be amplified (e.g, EGFR exon). In some embodiments the effective Tₘ of each primer is at least 10° higher than the Tₘ of the upstream excess primer to ensure that it binds first. In some embodiments, correct initiation of limiting primer strands is delayed one cycle.

In some embodiments, samples which find use with embodiments described herein include clinical samples, diagnostic samples, research samples, etc. In some embodiments, samples require processing by one or more techniques understood in the art prior to use in methods described herein.

In some embodiments one or more primer pairs (e.g., SEQ ID NOS.:1-8) are provided for the amplification of one or more target sequences (e.g., human EGFR exons 18, 19 20, and/or 21). In some embodiments, amplification (e.g., by LATE-PCR) of a sample nucleic acid (e.g., human genomic DNA, DNA obtained from a tumor, etc.) with one or more primer pairs (e.g., SEQ ID NOS.:1-8) produces one or more target amplicons (e.g., human EGFR exons 18, 19 20, and/or 21). In some embodiments, one or more probe sets (e.g., SEQ ID NOS.:9-13, SEQ ID NOS.:14-18, SEQ ID NOS.:19-22, and/or SEQ ID NOS.:23-29) are provided to interrogate one or more target amplicons (e.g., human EGFR exons 18, 19 20, and/or 21) for mutations in the target sequences. In some embodiments, each probe set is configured to interrogate on target amplicon. In some embodiments, each probe set is uniquely fluorescently labeled to allow differentiation between the probe sets. In some embodiments, each probe set comprises probes which hybridize to the entirety of one target amplicon. In some embodiments, probes from a probe set hybridize to adjacent sequences along a target amplicon, such that the entirety of the amplicon is interrogated for mutations. In some embodiments, each probe set comprises signaling and quencher probes according to embodiments described herein. In some embodiments, each probe set and the corresponding target amplicon provides a uniquely shaped fluorescence signature according the presence (and identity of) or absence of mutations within the target sequence. In some embodiment, analysis of the fluorescence signature for a probe set and target amplicon reveals the presence, location and identity of mutations contain in the target sequence. In some embodiments, each probe set is configured to detect, identify, and/or discriminate any mutation located within its corresponding target sequence.

### BRIEF DESCRIPTION OF THE DRAWINGS

FIG. 1, Panels A-D are schematics showing hybridization of two sets of signaling and quencher probes to a single-stranded nucleic acid target sequence in a sample as a function of temperature; and FIG. 1. Panel E, shows the fluorescence versus temperature of the sample.

FIG. 2, Panels A-D show the use of fluorescent signatures to detect the T790M mutation in EGFR exon 20 and the L858R mutation in EGFR exon 21 in the cancer cell line NCI-H1975.

FIG. 3, Panels A-C show the use of fluorescent signatures to detect and distinguish between various deletions in EGFR exon 19 in the cancer cell lines CRL-2868, CRL-2869, and CRL-2871.

### DETAILED DESCRIPTION

Provided herein are compositions (e.g., reagents, reactions mixtures, etc.), methods (e.g., research, screening, diagnostic), assays, and kits for obtaining rapid and reliable sequence analysis (e.g., identification of mutation in EGFR). In some embodiments, the methods, compositions, and kits provided herein provide diagnostically relevant information as well as a basis for treatment of patients who suffer from cancer, are at increased risk of developing cancer, or have had cancer in the past. EGFR is used as an example herein to illustrate certain embodiments. It should be understood that the methods and reagents described herein find more general use, and are not limited to the detection and analysis of EGFR sequences. In some embodiments, virtually any mutation in EGFR is identified by the compositions and methods provided herein. In some embodiments, assays provided herein utilize LATE-PCR (U.S. Pat. No. 7,198,897; incorporated herein by reference in its entirety) and/or Lights-On/Lights-Off probe sets (International Application No. PCT/US10/53569; incorporated herein by reference in its entirety) for identification of mutations in EGFR. When used together in single closed-tube reactions these techniques (LATE-PCR and Lights-On/Lights-Off) generate an exceptional amount of information about the mutational status of a gene target. In some embodiments, LATE-PCR amplifies four single-stranded amplicons, one for each of the exons 18, 19, 20, 21. In some embodiments, four sets of Lights-On/Lights-Off probes in four colors survey these amplicons for their sequences at every nucleotide. In some embodiments, each single-tube assay distinguishes wild type from all possible mutations. In some embodiments, the single-tube assay generates a different fluorescent signature for each mutation in EGFR. In some embodiments, the compositions and methods provided herein are capable of identifying the 4 major EGFR mutations, high frequency mutations, as well as low frequency mutations. In some embodiments, compositions and methods are automatable and suitable for use with standard PCR equipment or a dedicated instrument.

Provided herein are compositions (e.g., reagents, reactions mixtures, etc.), methods (e.g., research, screening, diagnostic), and systems (e.g., kits, data collection and analysis) for analysis of nucleic acid (e.g., human nucleic acid (e.g., DNA, RNA (e.g., human EGFR gene))). In particular, provided herein are compositions, methods, and systems that permit sensitive and specific detection of one or more mutations in the human EGFR gene (e.g., exon 18, exon 19, exon 20, exon 21). In some embodiments, method and compositions are provided for detection and/or identification of any mutation occurring in exons 18, 19, 20 and/or 21 of the human EGFR gene. Methods herein find use in identifying EGFR mutations in simple and complex samples, including samples containing nucleic acids from multiple cell types (e.g. cancerous and non-cancerous). In some embodiments, multiplex, single-tube reactions are provided that can simultaneously identify and distinguish multiple different EGFR mutations in complex samples using fast and efficient assays and detection equipment.

For example, provided herein is a set of single-tube homogeneous multiplexed assays for detection and analysis of EGFR mutations in exon 18, exon 19, exon 20, and/or exon 21. In some embodiments, assays and reagents provided herein detect and identify any mutations occurring along the length of exons 18-20 of human EGFR. In some embodiments, assays provided herein utilize LATE-PCR (U.S. Pat. No. 7,198,897; incorporated herein by reference in its entirety). PRIMESAFE II (PRIMESAFE is a trademark of Smiths Detection Inc.)(U.S. Patent Application No. 20080193934; incorporated herein by reference in its entirety), and Lights-On/Lights-Off probe sets (International Application No. PCT/US10/53569; incorporated herein by reference in its entirety).

In some embodiments, the methods, compositions, and kits provided herein produce fluorescence signatures that relate to the sequence of the probed region (e.g., exons 18-21 of human EGFR). In some embodiments, the fluorescence signature detects mutations in the probed region of the human EGFR gene. In some embodiments, assays provided herein determine whether a sample contains one or more EGFR mutations (e.g., in exons 18-21). In some embodiments, assays provided herein identify wild-type EGFR. In some embodiments, assays provided herein detect mutations in EGFR. In some embodiments, assays provided herein identify the location mutations in EGFR. In some embodiments, assays provided herein identify the location and identity (e.g., A to C, C to G, G to T, etc.) of mutations in EGFR. In some embodiments, assays provided herein differentiate a single mutation in the probed region from wild-type. In some embodiments, assays provided herein detect and identify a single mutation in the probed region. In some embodiments, assays provided herein differentiate between different mutations in the probed region (e.g., differentiating between single mutations at different locations in EGFR). In some embodiments, assays provided herein provide the location and/or identity of multiple mutations in EGFR (e.g., 2 mutations, 3 mutations, 4 mutations, 5 mutations, 6 mutations, 7 mutations, 8 mutations, 9 mutations, 10 mutations, 20 mutations, etc.). In some embodiments, multiple mutations within the same exon of EGFR (e.g., exon, 18, exon, 19, exon 20, exon 21, etc.) can be detected, identified, and/or differentiated with assays provided herein. In some embodiments, multiple mutations within the hybridization region of the same probe can be detected, identified, and/or differentiated with assays provided herein.

Compositions, kits, and methods provided herein provide sensitive and robust amplification starting with low initial numbers of target sequences (e.g. either absolute numbers or relative to non-target sequences). In some embodiments, amplified target sequences which are substantially longer than individual fluorescent hybridization probes are analyzed using sets of probes which use the same colored fluorophore. In some embodiments, silent/neutral mutations which do not cause resistance to cancer therapies or which do not affect the severity of the cancer are distinguished from mutations which do cause drug resistance or increased cancer severity (i.e. mutations of significant interest). In some embodiments, each of the different possible mutations of significant interest (e.g. mutations that result in drug resistance) is distinguished from the others. In some embodiments, mutations of significant interest are detected in sample mixtures comprising sequences lacking the mutations.

In some embodiments, signaling probes and quenching probes for use with EGFR mutation detection and identification assays are provided. Signaling probes and quenching probes are typically mismatch-tolerant. A mismatch-tolerant probe hybridizes in the assay, not only to a target sequence that is perfectly complementary to the probe, but also to variations of the target sequence that contain one or more mismatches due to substitutions, additions or deletions. For mismatch-tolerant probes, the greater the variation of the target from perfect complementarity, the lower the Tₘ of the probe-target hybrid. In some embodiments, sequence-specific probes are employed. A sequence-specific probe hybridizes in the assay only to a target sequence that is perfectly complementary to the probe (e.g. at a given temperature). In some embodiments, combinations of sequence-specific and mismatch-tolerant probes are employed in an assay. If a probe is sequence-specific, its lack of hybridization will be apparent in the melt curve and the derivative curve. For example, if a signaling probe hybridizes, causing an increase in fluorescence, but its associated quencher probe does not hybridize, fluorescence will not decrease as the temperature is lowered through the Tₘ of the quencher probe, revealing that the quencher probe did not hybridize and indicating a target mutation in the sequence complementary to the quencher probe. While this result indicates a mutation in the target sequence for the quencher probe, it does not allow for determination of which one of several possible mutations of that sequence is present. In some embodiments, it is preferable that the associated quencher probe be mismatch tolerant, if the assay is to provide differentiation of different mutations, distinguished by their different effects on the melting curve (and derivative curve) due to differing Tₘ effects of different mutations.

In some embodiments, a signaling probe of a set has a higher Tₘ with respect to the single-stranded nucleic acid target sequence than does its associated quencher probe. With that relationship, as a sample is subjected to melt analysis, for example, as temperature is increased signal first increases as the quencher probe melts off and then decreases as the signaling probe melts off. With the opposite relationship, signal remains quenched as the lower Tₘ signaling probe melts off and does not then increase as the higher Tₘ quencher probe melts off. The preferred relationship thus provides more information. In some embodiments, it is preferred that the quencher probe of a set reduces the signal from its associated signaling probe to a very large extent. In such embodiments, it is preferred that the concentration of the quencher probe equal or exceed the concentration of the signaling probe. In order to maximize signal amplitude, certain embodiments utilize probe concentrations that are in excess with respect to the single-stranded nucleic acid target sequence, thereby ensuring that all or nearly all copies of the target sequence will have hybridized probes.

Methods provided herein include the use of a single set of interacting signaling and quencher probes. Methods also include the use multiple sets of interacting signaling and quencher probes. In some embodiments in which multiple sets of interacting signaling and quencher probes are used, each signaling probe is detectably distinguishable from the others. In some embodiments in which multiple sets of interacting signaling and quencher probes are used, each signaling probe is not detectably distinguishable from the others. In some embodiments in which each signaling probe is not detectably distinguishable from the others, an overall fluorescence signature is produced by the multiple probes. In such embodiments, the location and identity of mutations is determined from changes to the overall fluorescence signature. Distinction of fluorescent probes may be by color (emission wavelength), by Tₘ, or by a combination of color and Tₘ. Multiple sets of interacting probes may be used to interrogate a single target sequence or multiple target sequences in a sample, including multiple target sequences on the same target strand or multiple target sequences on different strands. Multiplex detection of multiple target sequences may utilize, for example, one or more sets of signaling/quencher probes specific to each target sequence. In some embodiments, multiplex methods utilize a different fluorescent color for each target sequence. Certain embodiments utilize the same color for two different target sequences, available temperature space permitting.

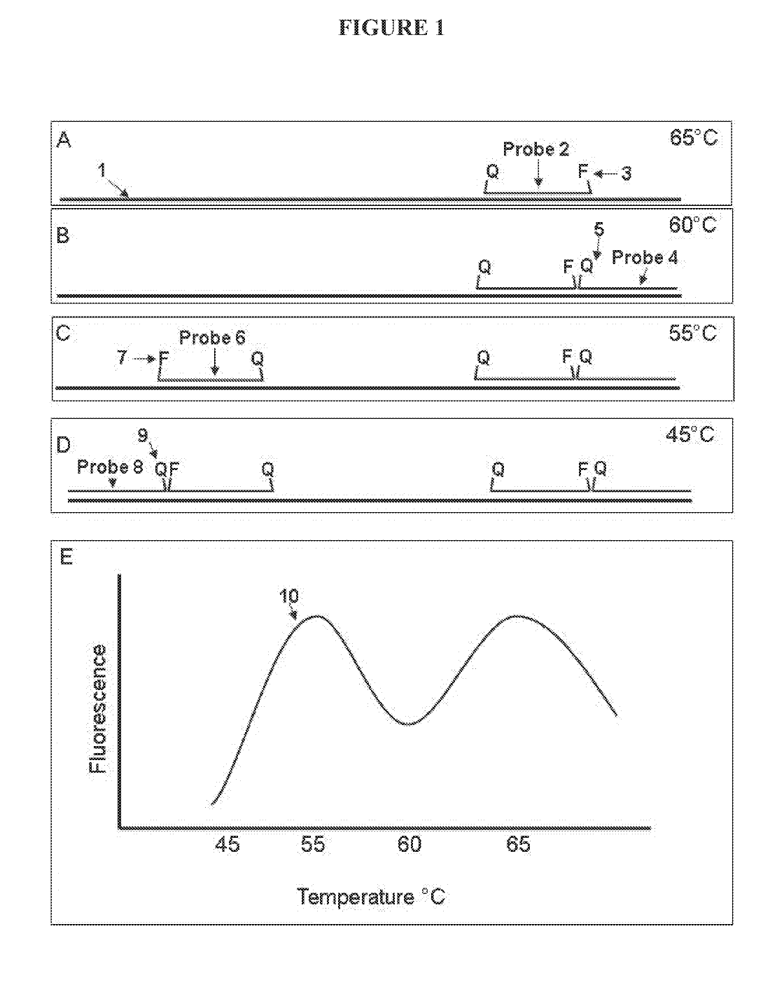

In some embodiments, methods comprise analyzing hybridization of signaling/quencher probe sets to one or more single-stranded EGFR nucleic acid target sequences as a function of temperature. Signal, preferably fluorescent signal, from the signaling probe or probes is acquired as the temperature of a sample is decreased (annealing) or increased (melting). In some embodiments, analysis includes acquisition of a complete annealing or melting curve, including both increasing and decreasing signals from each signaling probe, as is illustrated in FIG. 1, Panel E. Alternatively, in some embodiments, analysis is based only on signal increase or signal decrease. Analysis may utilize only signals at select temperatures rather than at all temperatures pertinent to annealing or melting. Analysis may include comparison of the hybridization of an unknown single-stranded nucleic acid target sequence to hybridization of known target sequences that have been previously established, for example, a compilation of melting curves for known mutations or combinations of mutations in EGFR or a table of digitized data for known mutations.

In some embodiments, analysis is performed to identify unknown or previously unidentified mutations. Assays provided herein are suitable for detecting and identifying unknown and/or previously unidentified mutations in EGFR (e.g., in exons 18-21 of human EGFR).

In methods provided herein, one or more single-stranded human EGFR nucleic acid target sequences to be analyzed may be provided by nucleic acid amplification, generally exponential amplification. Any suitable nucleic amplification method may be used. Preferred amplification methods are those that generate amplified product (amplicon) in single-stranded form so that removal of complementary strands from the single-stranded target sequences to be analyzed is not required. In some embodiments, amplification reactions are performed to produce separate amplicons for each strand of human EGFR (e.g., amplification reactions in separate vessels). In some embodiments, probe sets are included in amplification reaction mixtures prior to the start of amplification so that reaction vessels containing amplified product need not be opened. When amplification proceeds in the presence of probe sets, it is preferred that the system be designed such that the probes do not interfere with amplification. In some embodiments, an asymmetric PCR method or non-symmetric LATE-PCR method is utilized to generate single-stranded copies. In some embodiments, non-symmetric PCR is performed to generate single stranded amplicons for each strand of the EGFR gene, or a portion thereof (e.g., in separate reaction vessels). PCR amplification may be combined with reverse transcription to generate amplicons from RNA targets. For example, reverse transcription may be combined with LATE-PCR to generate DNA amplicons corresponding to RNA targets or the complements of RNA targets. In some embodiments, amplification methods that generate only double-stranded amplicons are not preferred, because isolation of target sequences in single-stranded form is required, and melt-curve analysis is more difficult with double-stranded amplicons due to the tendency of the two amplicons to collapse and eject hybridization probes. However, in some embodiments, amplification of double-stranded amplicons may be followed by amplification of just one strand via a two step method, for instance, by secondary addition of more of just one primer, or secondary addition of a new single to the reaction.

In some embodiments, methods provided herein do not utilize generation of detectable signal by digestion of signaling probes, such as occurs in 5′ nuclease amplification assays. In a PCR amplification reaction, for example, avoidance of probe digestion may be accomplished either by using probes whose Tm's are below the primer-extension temperature, by using probes such as those comprising 2′ O-methyl ribonucleotides that resist degradation by DNA polymerases, or by using DNA polymerases that lack 5′ exonuclease activity. Avoidance of probe interference with amplification reactions is accomplished by utilizing probes whose Tm's are below the primer-extension temperature such that the probes are melted off their complementary sequences during primer extension and, most preferably, during primer annealing, at least primer annealing after the first few cycles of amplification. For example, a LATE-PCR amplification method may utilize a two-step PCR with a primer-annealing/primer-extension temperature of 75° C. in the presence of a set of mismatch-tolerant molecular beacon probes having Tm's against the wild-type target sequence ranging from 75° C. to 50° C., which ensures that none of the probes interfere significantly with amplification of the target sequence.

In LATE-PCR amplification, for example, the Excess Primer strand is the single-stranded amplicon to which probe sets hybridize. It therefore is or contains the single-stranded nucleic acid sequence that is analyzed. Its 5′ end is the Excess Primer, and its 3′ end is the complement of the Limiting Primer. If the sequence to be analyzed lies between the Excess Primer and the Limiting Primer, the starting sequence that is amplified and the Excess Primer strand both contain that sequence. If in the starting sequence to be amplified the sequence desired to be analyzed includes a portion of either priming region, it is useful that the primer be perfectly complementary to that portion so that the Excess Primer strand contain the desired sequence. Primers need not be perfectly complementary to other portions of the priming regions. Certain embodiments of methods provide single-stranded nucleic acid target sequence to be analyzed by amplification reactions that utilize “consensus primers’ that are not perfectly complementary to the starting sequence to be amplified, and care is taken to ensure that the Excess Primer strand, which is or contains the single-stranded target sequence that is actually analyzed, contains the desired sequence.

In some embodiments, assays provided herein utilize PRIMESAFE II (described in U.S. Patent Application No. 20080193934; herein incorporated by reference in its entirety). PRIMESAFE II is a class of reagents added to PCR reactions to suppress mis-priming. PRIMESAFE II reagents are comprised of linear oligonucleotides that are chemically modified at their 5′ and or 3′ ends. In some embodiments, the assays described here make use of a formulation of PRIMESAFE II that has two strands, the first strand of which is modified at both the 5′end and the 3′end by covalent linkage of dabcyl moieties, the second strand of which is complementary to said first strand and is chemically modified by addition of dabcyl moieties at both the 5′end and the 3′end.

As one versed in the art will appreciate, some fluorescent thermocyclers have capacities for more than four fluorescent colors. It is contemplated that one or more additional colors could be utilized for amplification and detection of one or more additional amplicons that are detected with one or more additional probes or sets of probes. Such additional amplicons could be built into assays at the request of an end-user with a particular desired application. In some embodiments, amplicons detected or analyzed by other methods are multiplexed with the assays described herein. In some embodiments, such amplicons are analyzed by sequencing use a procedure such as “Dilute-N-Go” sequencing which is convenient for sequencing on or more strands of DNA generated in a multiplex LATE-PCR assay (Jia, Y., Osborne, A., Rice, J. E., and Wangh, L. J. (2010) Dilute-‘N’-Go Dideoxy Sequencing of All DNA Strands Generated in Multiplex LATE-PCR Assays, Nucleic Acids Research.; herein incorporated by reference in its entirety).

In some embodiments, a single target (e.g., exons 18, 19, 20, 21) is analyzed with multiple probe sets that together generate a composite fluorescent signals over a wide temperature range, the first derivative of said composite fluorescent signal is hereafter referred to as a temperature-dependent fluorescence signature. In some embodiments, more than one target is visualized using probe sets of the same color by designing the signals for one set of probes in a temperature range that is different from the temperature range for a separate target. In some embodiments, the signals from the two targets fuse into one composite temperature-dependent fluorescence signature which is informative as to the presence/absence of multiple mutations. In some embodiments, probe sets for each target in a multiplex assay are employed to achieve maximum coverage, as well as maximum resolution over the probed sequence.

The combination of LATE-PCR and Lights-On/Lights-Off probes lends itself to additional closed-tube strategies that do not require prior separation or purification of targets. In some embodiments, mixtures of targets can be diluted to generate replicate reactions each containing 10 or fewer molecules. In some embodiments, replicates that have only one sequence necessarily display the same fluorescent signature, while samples that contain mixtures of 1:10 to 10:1 molecules will have a different fluorescent signature. The initial dilution of a sample, that would render retrieved genomic material in proportions of 1:10 (the current detection sensitivity for Lights-On/Lights Off), allows the identification of a rarer alleles in a background of excess wild type DNA. In some embodiments, a tissue sample is taken apart mechanically, with or without guidance based on histological analysis of the sample, and one or more fractions of the sample are placed in multiple wells of a 96 well plate for analysis. This effectively reduces or eliminates the background of abundant wild type DNA in some fractions by isolating cells and letting their fluorescent signatures stand alone. By this mechanism cells which contain a rare mutation are identified when that cell is a single unit in a reaction well of a plate.

A second strategy for increasing the percentage of the desired target in a sample containing a substantial amount of nucleic acid from non-target sources (e.g., non-cancer cells, non-drug-resistant cells, etc.) makes use of either selective isolation of the target prior to amplification, or selective amplification of the target, or both. Selective isolation and/or amplification can increase the proportion of the desired target more than 2 fold, 5 fold, 10 fold, 20 fold, 100 fold, 1000 fold, 10,000 fold. Selective isolation of a specific sequence is well known in the art and typically involves sequence specific hybridization of the target to a solid matrix followed by washing and release. Selective amplification is also known in the art and includes methods known as Cold PCR, Ice Cold PCR which preferentially favor amplification of the desired target. An alternatively strategy uses blockers which preferentially reduce the efficiency of amplification of the undesired target (Provisional Patent Application No. 61/419,639; herein incorporated by reference in its entirety) by preventing priming of the undesired target. A third strategy uses blockers or probes which hybridized to the undesired target and inhibit primer extension along that target.

The embodiments provided herein are not limited by sample (e.g., tissue, cell, nucleic acid, DNA, etc.) processing or preparation techniques. Any suitable methods for sample preparation may find use with the compositions, kits, and methods described herein. In some embodiments, cell isolation techniques are utilized. In some embodiments, cells are purified, isolated, or sorted based on morphology or specific markers (e.g., cell-specific markers, cancer-specific markers, etc.). In some embodiments, microscopy (e.g., light microscopy, confocal microscopy, fluorescence microscopy, etc.) is utilized for cell sorting or for cell identification. In some embodiments, cells (e.g., cancer cells, cells of interest) are isolated from other sample material. In some embodiments, cells are mechanically separated from a tissue sample using any suitable technique (e.g., laser catapulting and laser capture dissecting microscope). In some embodiments, cells are mechanically separated from a tissue sample using a laser catapulting or laser capture dissecting microscope. By this method, cells that appear abnormal on a histology slide are harvested directly. Laser capture micro dissection allows the specific isolation of cells, by cutting away unwanted normal cells, and collecting only the abnormal cells of interest (Espina 2007; herein incorporated by reference in its entirety). The cells of interest are selected and then lifted from the slide the cells are placed on, and moved into a reaction tube (Emmert-Buck 1996; herein incorporated by reference in its entirety). Incorporating histology into the assay by laser micro-dissection provides a method that specifically selects for cells which are aberrant in morphology for further analysis. Further, selection of aberrant cells through micro dissection or other means circumvents detection limits.

### EXPERIMENTAL

Features and embodiments of methods provided herein are illustrated in the Examples set forth below in conjunction with the accompanying Figures. The Examples should be viewed as exemplary and not limiting in scope.

### Example 1

### Assay for Detection of Mutations in Exons 18-21 of EGFR

Experiments were conducted during development of embodiments of the present invention to develop an assay using LATE PCR and Lights-On/Lights-Off for scanning EGFR exons 18-21 for any and all possible mutations. Four amplicons are generated in the assay, one per exon, and each is surveyed with probes of a different fluorescent color. The assay is tested using genomic DNA from cell lines to detect the four most prevalent mutations in the EGFR oncogene. Each of the most common mutations, as well as many less common mutations, display a unique “fluorescent signature,” easily distinguished from that of the wild type EGFR sequence. Thus, in closed-tube reactions, the specific fluorescent signature analyzed in four colors defines the exact position and nucleotide composition of a particular mutation. In validating the assay, the exact nature of the DNA sequence that generates each fluorescent signature is confirmed the Dilute-‘N’-Go sequencing protocol.

Preparation of genomic DNA from cell lines is carried out as previously described, using QUANTILYSE (Pierce et al. 2002; herein incorporated by reference in its entirety). Exons 18-21 of the EGFR gene are amplified by LATE-PCR. Four sets of primers (e.g., SEQ ID NOS: 1-8), 1 pair for each exon, are used to generate the amplicons to be analyzed (See, e.g., Table 1).

TABLE 1

| SEQ ID NO. | Primer Name: | Tm: | Primer Sequence (5′-3′): |
|---|---|---|---|
| 1 | Limiting Primer | 75 | CCCAGAGGCCTGTGCCAGGGACCTTAC |
| 2 | Excess Primer | 72 | CTTGTCTCTGTGTTCTTGTCCCCCC |
| 3 | Limiting Primer | 75 | CCATGGACCCCCACACAGCAAAGCAGAAACTCAC |
| 4 | Excess Primer | 72 | GCCAGTTAACGTCTTCCTTCTCTCTCTGTCATA |
| 5 | Limiting Primer | 74 | TGGGAGCCAATATTGTCTTTGTGTTCCCGGACATAGT |
| 6 | Excess Primer | 72 | GTGCCTCTCCCTCCCTCCAG |
| 7 | Limiting Primer | 75 | AGGAAAATGCTGGCTGACCTAAAGCCACCTCCTTAC |
| 8 | Excess Primer | 72 | CTCACAGCAGGGTCTTCTCTGTTTCAG |

Primers are designed in accordance to LATE-PCR criteria (Pierce, K. E., et al. 2005, Sanchez, J. A., et al. 2004; herein incorporated by reference in their entirety). Two primers are used at unequal concentrations with different melting temperatures (Tₘ). The excess primer (EP) anneals at a lower Tₘ than the limiting primer (LP). The Tₘ of the LP is 3-5 degrees greater than that of the EP. The primer pairs each amplify their respective exon for both wild type and mutant targets. The primers are placed in intronic sequences (except in the case of EGFR exon 20 where the primers reside within the exon within a region with no reported mutations in NSCLC) as to equally amplify either wild type or mutant sequences. These primer locations allow the Taq DNA polymerase to amplify both mutant and wild type targets to equal efficiency. Consequently, the addition of PRIMESAFE II (PSII) is included to increase polymerase specificity.

The reaction conditions are in accordance to previously established LATE-PCR protocols. For example, 25 ul reactions are set up with 1.25 units of Taq, 10×Taq PCR reaction buffer, 3 mM Mg+, 1M EP, 50 nM LP, 400 nM dNTP, and PSII. Detection of the products and evaluation of the specificity of the amplification reaction is assessed initially at endpoint using 10×SYBR Green dye as a control. A thousand copies of each target are added into parallel reaction mixtures for each exon. The assay is performed with one exon per tube or in multiplex format.

Mutations in the EGFR amplicons are detected using Lights-On/Lights-Off probes (e.g., SEQ ID NOS: 9-29, e.g. See Table 2), and are based on the different fluorescent patterns (signatures) that are generated from the mutant targets when compared to the wild type samples.

TABLE 2

| SEQ ID NO. | Probe Name: | Tm: | Probe Sequence (5′-3′): | 5′ Modification | 3′ Modification |
|---|---|---|---|---|---|
| 9 | Exon 18 ON | 71 | GGTTGATCTTTTTGAATTCAGTTTCCTTCAAGA | FAM | BHQ-1 |
| 10 | Exon 18 OFF | 65 | CGGAGCCCAGCACT | BHQ-1 | BHQ-1 |
| 11 | Exon 18 ON | 69 | CAAAGAGAGCTTGGTTGGGAGCTTTG | BHQ-1 | FAM |
| 12 | Exon 18 ON | 59 | GGCTCCACTGGGTGTAAGCC | BHQ-1 | FAM |
| 13 | Exon 18 OFF | 61 | AGGCTCCACAAGCTG | BHQ-1 | C3 spacer |
| 14 | Exon 19 ON | 69 | GGACCTTCTGGGATCCAGAGTCCCCC | Cal Red 610 | BHQ-2 |
| 15 | Exon 19 OFF | 62 | ACGGGAATTTTAACTTTCTC | CS spacer | BHQ-2 |
| 16 | Exon 19 ON | 59 | CCGCTTTCGGAGATGTTGCTTGG | BHQ-2 | Cal Red 610 |
| 17 | Exon 19 OFF | 57 | CTCTTAATTTCTTGATAGCG | BHQ-2 | BHQ-2 |
| 18 | Exon 19 ON | 62 | TTATCGAGGATTTCCTTGTTGGAA | BHQ-2 | Cal Red 610 |
| 19 | Exon 20 ON | 59 | GCAGATACCCAGTAGGCGG | Quasar 670 | BHQ-2 |
| 20 | Exon 20 OFF | 55 | CTGCATGGTGAAGGTGAG | BHQ-2 | BHQ-2 |
| 21 | Exon 20 OFF | 64 | GCATGAGCCGCGTGATGAG | BHQ-2 | BHQ-2 |
| 22 | Exon 20 ON | 69 | CCAGGGGGCAGCCGAAGG | BHQ-2 | Quasar 670 |
| 23 | Exon 21 OFF | 50 | CCAGCATTATGGCTCGCCC | BHQ 1 | C3 spacer |
| 24 | Exon 21 ON | 70 | TTAAAATCTGTGATCTTGGCATGCTGCGGTGAA | Cal Orange 560 | BHQ-1 |
| 25 | Exon 21 ON | 56 | TTTTTGTCTCCCCCTGCATGGTATTCTTAA | BHQ-1 | Cal Orange 560 |
| 26 | Exon 21 OFF | 44 | TCTCTTCTGTACCC | BHQ-1 | C3 spacer |
| 27 | Exon 21 ON | 59 | CCACGGTCCCCCAAGTAGTTTATGCCGG | Cal Orange 560 | BHQ-1 |
| 28 | Exon 21 OFF | 42 | CTAGGTCTTGGTGGATTGAGCG | BHQ-1 | BHQ-1 |
| 29 | Exon 21 ON | 55 | CCCACCAGTATGTTCCTGGTTGGG | BHQ-1 | Cal Orange 560 |

Detection of mutations is determined at endpoint by the fluorescent pattern (signature) generated from the different mutant EGFR cell lines, Table 3.

TABLE 3
EGFR Cell Lines from the ATCC

| Cell Line | Mutation | Frequency |
|---|---|---|
| CRL-2868 | Δ E746-A750 | 45% |
| CRL-2869 | Δ E746-T751/S752I | 45% |
| CRL 2871 | Δ L747-E749/A750P | 45% |
| NCI-H3255 | L858R/amplification | 40% |
| NCI-H1975 | L858R and T790M | 45/50% |

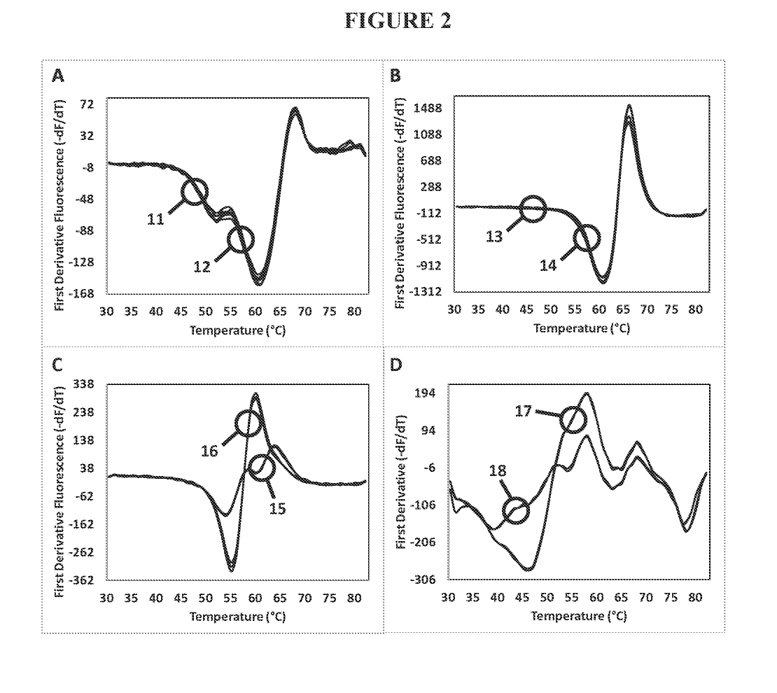

FIG. 2, Panels A-D show the fluorescent signatures for EGFR exons 18-21 from a control wild-type DNA sample and from the cancer cell line NCI-H1975 that is wild-type sequence for EGFR exon 18 and EGFR exon 19 but that carries the T790M mutation in EGFR exon 20 and the L858R mutation in EGFR exon 21. FIG. 2, Panel A, shows matching fluorescent signatures for EGFR exon 18 from the wild-type control (line 11) and the cancer cell line (line 12) indicating that the sequence of EGFR exon 18 from the cancer cell line is wild-type; FIG. 2, Panel B shows fluorescent signatures for EGFR exon 19 from the wild-type control (line 13) and the cancer cell line (line 14) indicating that the sequence of EGFR exon 19 from the cancer cell line is wild-type; FIG. 2, Panel C shows different fluorescent signals signatures for EGFR exon 20 from the wild-type control (line 15) and the cancer cell line (line 16); and FIG. 2, Panel D shows different fluorescent signals signatures for EGFR exon 21 from the wild-type control (line 17) and the cancer cell line (line 18). Differences between the wild-type and cancer cell line fluorescent signal signatures in EGFR exon 20 and EGFR exon 21 reveal the presence of the mutations in the cancer cell line.

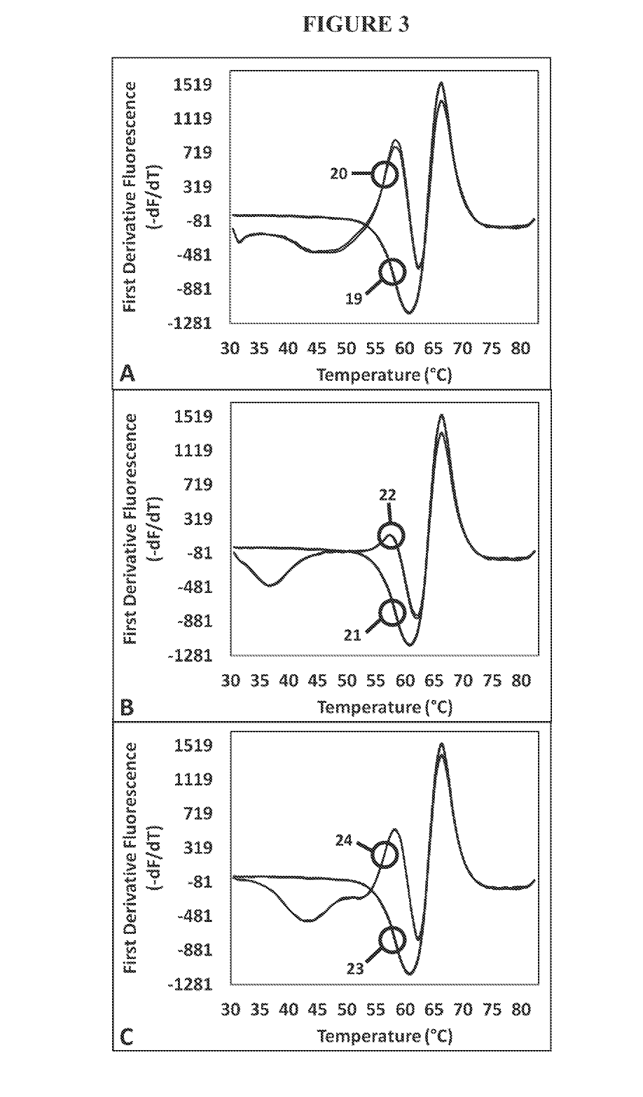

FIG. 3, Panels A-C show the fluorescent signal signatures for EGFR exon 19 from a control wild-type DNA sample and from different cancer cell lines with different deletions in EGFR exon 19 (see Table 3). FIG. 3, Panel A shows the differences in the fluorescent signals signatures for EGFR exon 19 from a wild-type control (line 19) and the cancer cell line CRL-2868 that carries the EGFR exon 19 deletion E746-A750 (line 20). FIG. 3, Panel B shows the differences in the fluorescent signals signatures for EGFR exon 19 from a wild-type control (line 21) and the cancer cell line CRL-2869 that carries the EGFR exon 19 deletion E746-T751 plus the point mutation S7521 (line 22). FIG. 3, Panel C shows the differences in the fluorescent signals signatures for EGFR exon 19 from a wild-type control (line 23) and from the cancer cell line CRL-2871 that carries the EGFR L747-E749 deletion plus the A750P point mutation (line 24). These data demonstrate that the assay identifies and distinguishes among various deletions in EGFR exon 19.

Controls include running parallel reactions for each exon using SYBR Green dye to determine the efficiency of the primers and to ensure that they generate the desired target. Control reactions with the primers in monoplex allow for both the quantification of material in the reaction and for the determination that each primer pair is capable of generating its target, demonstrating that each primer pair works before they are grouped into a single tube and run as a multiplex. Lights-On/Lights-Off probes for each exon are run in parallel as monoplex reactions to establish signature patterns for wild type exons as well as mutant exons. The controls for the probes in monoplex ensure that all the probes bind their target and give a signal. When the probes come across a mutant target, any failure to fluoresce or a decrease in fluorescence is directly attributable to the mutation that prevents probe from binding, and therefore indicates that a mutant protein sequence is present.

A verification study is performed to attest to the repeatability and limits of detection of the assay. The reproducibility of the assay is determined by repeated blinded studies. The limits of detection for the assay are determined by the diagnostic sensitivity and specificity of the assay. The specificity is measured as the known mutated samples that are detected in this assay, while the specificity measures the proportion of wild type samples that are tested and recorded as wild type.

Verification studies for the LATE PCR Lights-On/Lights-Off assay are conducted using cell lines. The cell lines used are found in Table 3. Mutations with various deletion characteristics within EGFR exon 19 as well as lower frequency mutations in EGFR exon 18 are used to study the limits of the assay, and to mimic scenarios which may appear in the clinical setting. Frozen and paraffin embedded samples are also used to verify the assay because these are the most likely to be used in the clinical setting. Frozen tissue provides a mimic to fresh tissue since it is not preserved with harsh chemical additives. Formalin fixed paraffin imbedded samples (FFPE) are the tissues which are used for retrospective studies as well as translational studies in cancer. The second advantage to using FFPE samples will be the ability to evaluate the Lights-On/Lights-Off system with the quality of DNA extracted from this archiving mechanism. Many of the fixative techniques used for FFPE damage the DNA which can make PCR difficult. Therefore these samples are able to test the robustness of the primers and the ability of the primer to amplify material which may not be of the highest quality. Conventionally, it has been difficult to amplify DNA that has been formalin-fixed and paraffin-embedded however current clinical methods for DNA preparation from FFPE samples have proven adequate for the retrieval of amplifiable DNA from these samples.

Controls for these verification studies include the use of the cell lines from the development portion of the assay run in parallel with the new cells lines and FFPE tissue, any changes in the way these probes act will be a function of the variability of the DNA with the way in which the probe signatures identify the alterations.

This verification study provides the limits of detection for the EGFR mutation detection assay. The different sources of DNA demonstrate how the different qualities in DNA effect mutation identification. The sensitivity of this assay is 5%-10% which is the current limit of detection for Lights-On/Lights Off probes. This means that at least 5%-10% of a sample or biopsy would have to contain mutant sequence in order to be detected. This limit of detection is due to the fact that the primers for this region will amplify both the wild type and mutant sequence at equal efficiency since there is no preference for a mutant sequence over a wild type sequence.

All publications and patents mentioned in the present application are herein incorporated by reference in their entireties. Various modification, recombination, and variation of the described features and embodiments will be apparent to those skilled in the art without departing from the scope and spirit of the invention. Although specific embodiments have been described, it should be understood that the claims should not be unduly limited to such specific embodiments. Indeed, various modifications of the described modes and embodiments that are obvious to those skilled in the relevant fields are intended to be within the scope of the following claims, and can be made without departing from the inventive concepts described herein.

### REFERENCES

Emmert-Buck, M. R., et al., Laser capture microdissection. Science, 1996. 274(5289): p. 998-1001.
Espina V, H. M., Pierobon M, Liotta L A, “Laser capture micro-dissection technology”. Expert Rev. Mol. Diagn., 2007. 7(5): p. 647-657.
Fan, Z., et al., Antitumor effect of anti-epidermal growth factor receptor monoclonal antibodies plus cis-diamminedichloroplatinum on well established A431 cell xenografts. Cancer Res, 1993. 53(19): p. 4637-42.
Gazdar, A. F., Activating and resistance mutations of EGFR in non-small-cell lung cancer: role in clinical response to EGFR tyrosine kinase inhibitors. Oncogene, 2009. 28 Suppl 1: p. S24-31.
Herbst, R. S., Review of epidermal growth factor receptor biology. Int J Radiat Oncol Biol Phys, 2004. 59(2 Suppl): p. 21-6.
Herbst, R. S., J. V. Heymach, and S. M. Lippman, Lung cancer. N Engl J Med, 2008. 359(13): p. 1367-80.
Kitamura, A., et al., Immunohistochemical detection of EGFR mutation using mutation-specific antibodies in lung cancer. Clin Cancer Res. 16(13): p. 3349-55.
Kobayashi, S., et al., EGFR mutation and resistance of non-small-cell lung cancer to gefitinib. N Engl J Med, 2005. 352(8): p. 786-92.
Kondo. I. and N. Shimizu, Mapping of the human gene for epidermal growth factor receptor (EGFR) on the p13 leads to q22 region of chromosome 7. Cytogenet Cell Genet, 1983. 35(1): p. 9-14.
Ludwig, J. A. and J. N. Weinstein, Biomarkers in cancer staging, prognosis and treatment selection. Nat Rev Cancer, 2005. 5(11): p. 845-56.
Lynch, T. J., et al., Activating mutations in the epidermal growth factor receptor underlying responsiveness of non-small-cell lung cancer to gefitinib. N Engl J Med, 2004. 350(21): p. 2129-39.
Maheswaran, S., et al., Detection of mutations in EGFR in circulating lung-cancer cells. N Engl J Med, 2008. 359(4): p. 366-77.
Mitsudomi, T., T. Kosaka, and Y. Yatabe, Biological and clinical implications of EGFR mutations in lung cancer. Int J Clin Oncol, 2006. 11(3): p. 190-8.
Mok, T. S., et al., Gefitinib or carboplatin-paclitaxel in pulmonary adenocarcinoma. N Engl J Med, 2009. 361(10): p. 947-57.
Paez, J. G., et al., EGFR mutations in lung cancer: correlation with clinical response to gefitinib therapy. Science, 2004. 304(5676): p. 1497-500.
Pierce, K. E., et al., QuantiLyse: reliable DNA amplification from single cells. Biotechniques. 2002. 32(5): p. 1106-11.
Rosell, R., et al., Screening for epidermal growth factor receptor mutations in lung cancer. N Engl J Med, 2009. 361(10): p. 958-67.
Sanchez, J. A., et al., Linear-after-the-exponential (LATE)-PCR: an advanced method of asynmetric PCR and its uses in quantitative real-time analysis. Proc Natl Acad Sci USA, 2004. 101(7): p. 1933-8.
Sorscher, S. M., EGFR mutations and sensitivity to gefitinib. N Engl J Med, 2004. 351(12): p. 1260-1; author reply 1260-1.
Wang, K., et al., Epidennal growth factor receptor-deficient mice have delayed primary endochondral ossification because of defective osteoclast recruitment. J Biol Chem, 2004. 279(51): p. 53848-56.
Yamazaki, H. et al., Amplification of the structurally and functionally altered epidermal growth factor receptor gene (c-erbB) in human brain tumors. Mol Cell Biol, 1988. 8(4): p. 1816-20.
Zhang, X., et al., An allosteric mechanism for activation of the kinase domain of epidermnnal growth factor receptor. Cell, 2006. 125(6): p. 1137-49.

1.-76. (canceled)

77. A method for detecting mutations in the human EGFR gene, comprising
a) providing:
i) a sample suspected of containing the human EGFR gene, or a portion of the human EGFR gene, and
ii) detection reagents comprising at least one pair of primers and at least one detectably distinguishable probe set of two or more hybridization probes which hybridize to adjacent target nucleic acid sequences in the human EGFR gene, each probe set comprising:
A) one or more quencher probe(s) labeled with a non-fluorescent quencher, and
B) one or more signaling probes labeled with a fluorescence-emitting moiety and a non-fluorescent quencher, wherein said signaling probes do not emit fluorescence above background when not hybridized to the target sequence, but emit a fluorescence signal above background upon hybridization to the target sequence in the absence of adjacently bound quencher probe, wherein, if both a signaling probe and an adjacently-bound quencher probe are hybridized to their target nucleic acid sequences, the non-fluorescent quencher of the quencher probe quenches the signal from the signaling probe;
b) amplifying all or a portion of said human EGFR gene with said primers;
c) detecting the fluorescence of said fluorescence-emitting fluorophore from each detectably distinguishable probe set over a range of temperatures;
d) generating temperature-dependent composite fluorescence curves or first derivative fluorescent signatures for each fluorescence-emitting fluorophore; and
e) analyzing said temperature-dependent fluorescence curves or first derivative fluorescent signatures to detect the presence or absence of one or more mutations in the human EGFR gene. Different degree of complementarity of at least one probe to a given mutation in the EGFR target sequence result in different temperature-dependent fluorescent signatures generated by said probe set and said target sequences that are used to differentiate different mutations in the EGFR gene.

78. The method of claim 77, wherein primers and probe sets are configured to hybridize to exon 18, exon 19, exon 20, or exon 21 of the human EGFR gene.

79. The method of claim 77, wherein each of said two or more probe sets are detectably distinguishable from all other probe sets in said detection reagents by (1) melting temperature, (2) emission wavelength of said fluorescence-emitting fluorophore, (3) rate of hybridization or melting, or (4) a combination thereof.

80. The method of claim 77, wherein said each probe set comprises signaling and quencher probes configured to span, coat, or tile a target sequence.

81. The method of claim 77, wherein said temperature-dependent fluorescence signature are derived from a composite melt curve or a composite annealing curve.

82. The method of claim 77, wherein the analyzing said temperature-dependent fluorescence signature comprises comparison to a previously established fluorescent signature.

83. The method of claim 77, wherein the analysis is performed by a computer.

84. The method of claim 77, wherein the target strand is a single-stranded nucleic acid.

85. The method of claim 77, wherein said target strands are generated by LATE-PCR amplification.

86. A reagent kit for identifying one or more mutations in the human EGFR gene comprising:
a) at least one pair of primers, wherein said primers are configured to bind to regions of the human EGFR gene, and wherein said primers are configured to amplify an intervening target region of the human EGFR gene; and
b) at least one probe set of two or more hybridization probes which hybridize to adjacent sequences along the length of the target region of the human EGFR gene, comprising:
i) at least one quencher probe labeled with a non-fluorescent quencher, and
ii) at least one signaling probe labeled with a fluorescence-emitting fluorophore and a non-fluorescent quencher, wherein said signal probe does not emit fluorescence above background when not hybridized to its target sequence, but emits a fluorescence signal above background upon hybridization to its target sequence in the absence of bound quencher probe, wherein, if both signaling and quencher probes are hybridized to adjacent target nucleic acid sequences, the non-fluorescent quencher of the quencher probe quenches the signal from the signaling probe.

87. The reagent kit of claim 86, wherein primers and probe sets are configured to hybridize to exon 18, exon 19, exon 20, or exon 21 of the human EGFR gene.

88. The reagent kit of claim 86, wherein each of said probe sets are detectably distinguishable from all other probe sets in said detection reagent kit by (1) melting temperature, (2) emission wavelength of said fluorescence-emitting fluorophore, or (3) a combination thereof.

89. The reagent kit of claim 88, wherein said each probe set comprises signaling and quencher probes configured to span or coat a target sequence.

90. The reagent kit of claim 86, wherein said primers are provided in the proper ratio for amplification by LATE-PCR.

91. The reagent kit of claim 86, further comprising one or more additional oligonucleotides to improve amplification reproducibility, to suppress mis-priming during amplification reactions, and/or to disrupt structural elements within target nucleic acid sequences during amplification reactions.


SEQUENCE LISTING (29 sequences)

SEQ ID NO: 1 - DNA, 27 bp, Artificial Sequence
  Synthetic
cccagaggcctgtgccagggaccttac

SEQ ID NO: 2 - DNA, 25 bp, Artificial Sequence
  Synthetic
cttgtctctgtgttcttgtcccccc

SEQ ID NO: 3 - DNA, 34 bp, Artificial Sequence
  Synthetic
ccatggacccccacacagcaaagcagaaactcac

SEQ ID NO: 4 - DNA, 33 bp, Artificial Sequence
  Synthetic
gccagttaacgtcttccttctctctctgtcata

SEQ ID NO: 5 - DNA, 37 bp, Artificial Sequence
  Synthetic
tgggagccaatattgtctttgtgttcccggacatagt

SEQ ID NO: 6 - DNA, 20 bp, Artificial Sequence
  Synthetic
gtgcctctccctccctccag

SEQ ID NO: 7 - DNA, 36 bp, Artificial Sequence
  Synthetic
aggaaaatgctggctgacctaaagccacctccttac

SEQ ID NO: 8 - DNA, 27 bp, Artificial Sequence
  Synthetic
ctcacagcagggtcttctctgtttcag

SEQ ID NO: 9 - DNA, 34 bp, Artificial Sequence
  Synthetic
ggttgatctttttgaattcagtttccttcaagat

SEQ ID NO: 10 - DNA, 14 bp, Artificial Sequence
  Synthetic
cggagcccagcact

SEQ ID NO: 11 - DNA, 26 bp, Artificial Sequence
  Synthetic
caaagagagcttggttgggagctttg

SEQ ID NO: 12 - DNA, 20 bp, Artificial Sequence
  Synthetic
ggctccactgggtgtaagcc

SEQ ID NO: 13 - DNA, 15 bp, Artificial Sequence
  Synthetic
aggctccacaagctg

SEQ ID NO: 14 - DNA, 26 bp, Artificial Sequence
  Synthetic
ggaccttctgggatccagagtccccc

SEQ ID NO: 15 - DNA, 20 bp, Artificial Sequence
  Synthetic
acgggaattttaactttctc

SEQ ID NO: 16 - DNA, 23 bp, Artificial Sequence
  Synthetic
ccgctttcggagatgttgcttgg

SEQ ID NO: 17 - DNA, 20 bp, Artificial Sequence
  Synthetic
ctcttaatttcttgatagcg

SEQ ID NO: 18 - DNA, 24 bp, Artificial Sequence
  Synthetic
ttatcgaggatttccttgttggaa

SEQ ID NO: 19 - DNA, 19 bp, Artificial Sequence
  Synthetic
gcagatacccagtaggcgg

SEQ ID NO: 20 - DNA, 18 bp, Artificial Sequence
  Synthetic
ctgcatggtgaaggtgag

SEQ ID NO: 21 - DNA, 19 bp, Artificial Sequence
  Synthetic
gcatgagccgcgtgatgag

SEQ ID NO: 22 - DNA, 18 bp, Artificial Sequence
  Synthetic
ccagggggcagccgaagg

SEQ ID NO: 23 - DNA, 19 bp, Artificial Sequence
  Synthetic
ccagcattatggctcgccc

SEQ ID NO: 24 - DNA, 33 bp, Artificial Sequence
  Synthetic
ttaaaatctgtgatcttggcatgctgcggtgaa

SEQ ID NO: 25 - DNA, 30 bp, Artificial Sequence
  Synthetic
tttttgtctccccctgcatggtattcttaa

SEQ ID NO: 26 - DNA, 14 bp, Artificial Sequence
  Synthetic
tctcttctgtaccc

SEQ ID NO: 27 - DNA, 28 bp, Artificial Sequence
  Synthetic
ccacggtcccccaagtagtttatgccgg

SEQ ID NO: 28 - DNA, 22 bp, Artificial Sequence
  Synthetic
ctaggtcttggtggattgagcg

SEQ ID NO: 29 - DNA, 24 bp, Artificial Sequence
  Synthetic
cccaccagtatgttcctggttggg